(19) United States
(12) Patent Application Publication
(10) Pub. No.: US 2012/0046671 A1
(43) Pub. Date: Feb. 23, 2012

(54) REPETITIVE CLIPPING TREATMENT DEVICE

(76) Inventors: Yoshiaki Matsuoka, Kanagawa (JP); Takayuki Iida, Kanagawa (JP); Ryousuke Oosako, Kanagawa (JP)

(21) Appl. No.: 13/318,759

(22) PCT Filed: May 12, 2010
(86) PCT No.: PCT/JP2010/058029

§ 371 Date: Nov. 3, 2011

(30) Foreign Application Priority Data
May 13, 2009 (JP) 2009-116544

(51) Int. Cl. A61B 17/10

(52) U.S. Cl. 606/143

(57) ABSTRACT

A repetitive clipping treatment device has a cylindrical sheath loaded with a clip series that has plural clips, a manipulating wire to move the clip series in the longitudinal direction of the sheath inside the sheath, a handle main body connected to the sheath and having the manipulating wire arranged therein, a slider to move the manipulating wire in the axial direction of the handle main body, and a slider displacement regulating member to regulate the displacement of the slider in the axial direction to different displacements required for clipping treatment of each clip of the clip series. The plural different displacements corresponding to the respective clips are determined based on the lengths to protrude and the retraction amounts of the respective clips.

### TECHNICAL FIELD

The present invention relates to a repetitive clipping treatment device which manipulates plural clips for endoscope treatment with a manipulating wire, to perform clipping treatment, such as stopping bleeding, closure of an affected part, such as a wound, or the like, in a living body, and particularly, to a repetitive clipping treatment device which can suppress the influence of retraction of a manipulating wire when clipping treatment using the manipulating wire is performed.

### BACKGROUND ART

In recent years, a clipping treatment device for an endoscope has been used which causes a clip to protrude from the forward end of an endoscope inserted into a living body to pinch a bleeding portion or a portion to be treated after removal of a diseased tissue with the clip, thereby stopping bleeding or performing suturing or closure of a wound.

In this clipping treatment device for an endoscope, clips are loaded into a sheath and protruding of a clip and pinching of a portion to be treated with the clip is performed by advancing or retreating a manipulating wire.

However, since the manipulating wire is thin compared to the inner diameter of the sheath, a relatively large space opens between the sheath and the manipulating wire. For this reason, when the sheath is curved during use of an endoscope, since the manipulating wire passes through the outside of the inner surface of the curved sheath due to this space, a difference occurs in path length between the sheath and the manipulating wire. For this reason, for example, when the sheath is curved after the manipulating wire is made to protrude by a predetermined amount from the forward end of the sheath with a state where the sheath is extended straightly being a reference state, so-called retraction occurs where the forward end of the manipulating wire is located inside the forward end of the sheath.

Thus, Patent Literature 1 suggests a treatment device for an endoscope having a flexible sheath which is passed through a treatment device guide pipe of the endoscope, a forward end treatment member which protrudes or retracts from a forward end portion of the flexible sheath, a manipulating wire which has a forward end connected to the forward end treatment member and is slidably passed through the flexible sheath in the axial direction, a first manipulating member which is connected to the proximal end of the flexible sheath, a second manipulating member connected to the proximal end of the manipulating wire. In the treatment device for an endoscope of this Patent Literature 1, the manipulation of relatively moving the first manipulating member and second manipulating member in the axial direction of the flexible sheath and the manipulating wire, whereby the flexible sheath and the manipulating wire slide relatively in the axial direction and the forward end treatment member protrudes or retracts from the forward end portion of the flexible sheath. The treatment device for an endoscope of Patent Literature 1 performs clipping one by one.

The treatment device for an endoscope of Patent Literature 1 provides a connection positioning means capable of changing one of the connection position of the proximal end of the flexible sheath to the first manipulating member and the connection position of the proximal end of the manipulating wire to the second manipulating member freely in the axial direction. The relationship in relative length between the manipulating wire and the flexible sheath can be arbitrarily adjusted by this connection positioning means. That is, the relative position between the manipulating wire and the flexible sheath can be arbitrarily adjusted. For this reason, in the treatment device for an endoscope of Patent Literature 1, a user of the treatment device for an endoscope can arbitrarily adjust the relative length relationship between the manipulating wire and the flexible sheath if needed to set and use the forward end treatment member in an optimal state at the forward end portion of the flexible sheath.

### CITATION LIST

### Patent Literature

Patent Literature 1: JP 2007-244826 A

### SUMMARY OF INVENTION

### Technical Problems

The treatment device for an endoscope of Patent Literature 1 performs one clipping at a time, although a user of the treatment device for an endoscope can arbitrarily perform the adjustment if needed to set and use the forward end treatment member in an optimal state at the forward end portion of the flexible sheath.

Here, in a repetitive clipping treatment device continuously using plural clips, even in an optimal state for a first clip, there is a problem that the remaining clips cannot be set in an optimal state due to influence of the above-mentioned retraction.

For this reason, in Patent Literature 1, as for one clip, the forward end treatment member can be set and used in an optimal state at the forward end portion of the flexible sheath. However, in the repetitive clipping treatment device using plural clips continuously, there is a problem that each clip cannot necessarily be set in an optimal state at the forward end portion of the flexible sheath.

The object of the invention is to solve the problems based on the conventional technique, and provide a repetitive clipping treatment device which suppresses the influence of retraction of a manipulating wire.

### Solution to Problems

To achieve the aforementioned objective, according to an aspect of the present invention, there is provided a repetitive clipping treatment device comprising a clip series made up of plural clips connected by a forward end of a subsequent clip being engaged with a rear end of a previous clip, and a connection member connected to the rearmost clip, a cylindrical sheath loaded with the clip series, a manipulating wire movably arranged in the sheath, and having a forward end detachably connected to the connection member so as to move the clip series in the longitudinal direction of the sheath in the sheath, a handle main body connected to the sheath and having the manipulating wire extending from the sheath arranged therein, a slider mounted on the outer periphery of the handle main body so as to be movable in the axial direction of the handle main body, engaged with the manipulating wire, and moving the manipulating wire in the axial direction of the handle main body, and a slider displacement regulating member mounted on the outer periphery of the handle main body so as to be rotatable in the circumferential direction of the handle main body and regulating the displacement of the slider in the axial direction of the handle main body to plural different displacements, required for clipping treatment of each clip of the clip series, wherein the plural different displacements regulated by the slider displacement regulating member and corresponding to the respective clips is calculated by obtaining an amount of retraction at the position of a clip of the clip series in advance, and determining the displacement on the basis of the length required to cause each clip to protrude, and the respective retraction amounts in each clip.

It is preferred that an engagement portion between the previous clip and the subsequent clip in the clip series is covered with a connection ring fitted so as to be able to advance or retreat with respect to the clips, and the connection ring is pressed against the inner surface of the sheath and closed inward in the sheath, and presses and retains at least one of the clips connected in the connection ring, and at least two or more skirt portions opened more widely than the inner diameter of the sheath after passing through a forward end of the sheath to prevent retreating to the inside of the sheath are provided in the circumferential direction of the connection ring at the same position in a movement direction of the clips.

Further, it is preferred that an extension portion is provided as a portion of the connection ring on the proximal end side and extends, and the extension portion prevents the clip and the connection ring from coming off of the forward end of the sheath during clipping treatment.

Further, it is preferred that the slider displacement regulating member has plural position regulating grooves which is formed according to the plural different displacements, respectively, at predetermined intervals in the circumferential direction of the handle main body, and have different positions at forward end portions thereof on the sheath side.

Further, it is preferred that the slider displacement regulating member has position regulating grooves equal to or more than the number of the clips provided in the clip series.

Further, it is preferred that the slider displacement regulating member is arranged so that a position regulating groove whose sheath-side forward end portion is nearest to the sheath, and a position regulating groove whose forward end portion is farthest from the sheath are adjacent to each other, and wherein a position regulating groove formed on the side where the position regulating groove whose forward end portion is nearest to the sheath, and the position regulating groove whose forward end portion is farthest from the sheath are not adjacent to each other is formed so that the forward end portion thereof is located at a position near the sheath gradually, as it goes from the position regulating groove whose forward end portion is nearest to the sheath, and the position regulating groove whose forward end portion is farthest from the sheath, on the side where the position regulating groove whose forward end portion is nearest to the sheath, and the position regulating groove whose forward end portion is farthest from the sheath are not adjacent to each other.

Further, it is preferred that the position regulating groove whose forward end portion is nearest to the sheath in the slider displacement regulating member is used for loading of the clip series into the sheath.

Further, it is preferred that the slider has a guide pin which extends toward the center of the handle main body and has a diameter capable of being inserted through the position regulating grooves, and when the position of one position regulating groove of the plural position regulating grooves overlaps the position of the guide pin, the slider (the slider) moves in the axial direction of the handle main body along the overlapped position regulating groove.

Further, it is preferred that the clip series includes three clips, and the slider displacement regulating member has the position regulating grooves at intervals of 90° in the circumferential direction of the handle main body.

Also, it is preferred that the clip has at least a claw portion and an arm portion, and the arm portion has a projection. In this case, the projection is wider than the arm portion.

### Advantageous Effects of Invention

According to the invention, plural different displacements of the slider corresponding to plural clips is calculated by obtaining an amount of retraction at the position of a clip of the clip series in advance, and determining the displacement on the basis of the length required to cause each clip to protrude, and the respective retraction amounts in each clip. For this reason, even if the sheath is curved, the influence of retraction of the manipulating wire can be suppressed, and a clip can be reliably caused to protrude from the forward end of the sheath during clipping treatment. As a result, the clipping treatment can be ensured.

### BRIEF DESCRIPTION OF DRAWINGS

FIG. 1 is a schematic perspective view illustrating a repetitive clipping treatment device of an embodiment of the invention.

FIG. 2A is a schematic cross-sectional view illustrating the configuration of a forward end portion of the repetitive clipping treatment device of the embodiment of the invention, and FIG. 2B is a schematic cross-sectional view as seen from an angle different from FIG. 2A by 90°.

FIG. 3 is a perspective view illustrating the schematic configuration of a clip used for a repetitive clipping treatment device of the embodiment of the invention.

FIG. 4A is a front view illustrating an example of a connection ring used together with a clip of the repetitive clipping treatment device of the embodiment of the invention, FIG. 4B is a cross-sectional view of the connection ring illustrated in FIG. 4A, and FIG. 4C is a bottom view of the connection ring illustrated in FIG. 4A.

FIG. 5 is a schematic cross-sectional view illustrating a handle portion of a manipulation handle of the repetitive clipping treatment device of the embodiment of the invention.

FIG. 6 is a schematic perspective view illustrating a main body rail of the manipulation handle of the repetitive clipping treatment device of the embodiment of the invention.

FIG. 7 is a schematic perspective view illustrating a home position mark of the handle portion of the manipulation handle of the repetitive clipping treatment device of the embodiment of the invention.

FIG. 8A is a schematic perspective view illustrating a slider guide of the manipulation handle of the repetitive clipping treatment device of the embodiment of the invention, and FIG. 8B is a schematic developed view illustrating a guide portion of the manipulation handle.

FIG. 9 is a schematic perspective view illustrating a rotating position regulating member of the handle portion of the manipulation handle of the repetitive clipping treatment device of the embodiment of the invention.

FIG. 10 is a graph illustrating differences in the retraction amount depending on the position of a clip, with the retraction amount on the vertical axis and the position of a clip on the horizontal axis.

FIG. 11 is a schematic developed view illustrating the slider guide of the repetitive clipping treatment device of the embodiment of the invention.

FIGS. 12A to 12G are schematic cross-sectional views illustrating a gradual state in the clipping manipulation of the repetitive clipping treatment device of the embodiment of the invention.

FIG. 13A is a schematic view illustrating an example of an adjusting mechanism of a manipulating wire of the handle portion of the manipulation handle of the repetitive clipping treatment device of the embodiment of the invention, FIG. 13B is a plan view illustrating the example of the adjusting mechanism of the manipulating wire of the handle portion of the manipulation handle of the repetitive clipping treatment device of the embodiment of the invention, and FIG. 13C is a cross-sectional view taken along the line G-G of FIG. 13B.

### DESCRIPTION OF EMBODIMENTS

Below, a repetitive clipping treatment device of the invention illustrated in the accompanying drawings will be described in detail on the basis of embodiments.

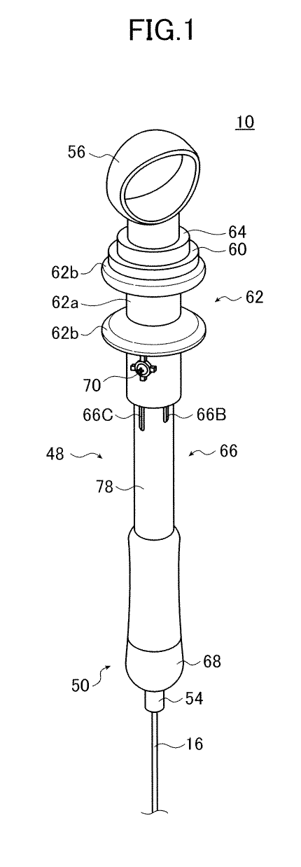

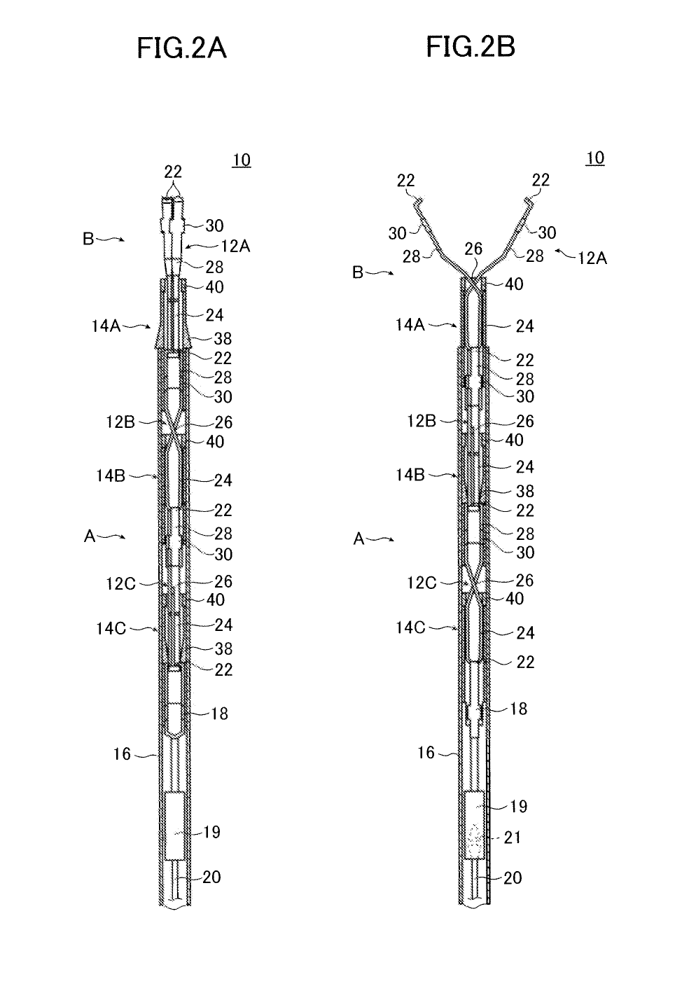

FIG. 1 is a schematic perspective view illustrating a repetitive clipping treatment device of an embodiment of the invention. FIG. 2A is a schematic cross-sectional view illustrating the configuration of a forward end portion of the repetitive clipping treatment device of the embodiment of the invention, and FIG. 2B is a schematic cross-sectional view as seen from an angle different from FIG. 2A by 90°.

The repetitive clipping treatment device (hereinafter referred to as a clipping treatment device) 10 of this embodiment is a repetitive type using plural clips 12. For example, a clip series A (refer to FIGS. 2A and 2B) which includes three clips 12 (12A, 12B, and 12C), a dummy clip 18 connected to the rearmost clip 12C among the clips 12A to 12C, and a connecting member 19 connected to the dummy clip 18 is housed at a forward end portion of a sheath 16 of a manipulation handle 48 (hereinafter, referred to as a manipulation handle 48) for a clipping treatment device.

In addition, the forward end portion is a forward end portion inserted into a living body when a treatment using the clipping treatment device 10 is performed using an endoscope. Hereinafter, the side where the forward end portion is present is referred to as the forward end side, and the side opposite to the forward end portion is referred to as a proximal end side (a manipulation handle 48 side).

In this embodiment, the manipulation handle 48 has a manipulating portion 50, a sheath 16, a manipulating wire 20 (refer to FIGS. 2A and 2B), or the like.

The sheath 16 is an ordinary long tubular sheath used in the clipping treatment device for an endoscope, for example, is a flexible coil sheath in which a metal wire is tightly wound. Although the sheath 16 will be described below in detail, the sheath has the clips 12 movably fitted on the forward end side therein, houses the manipulating wire 20 connected to the clip 12 via the dummy clip 18 and the connecting member 19, and is connected to the manipulating portion 50 on the proximal end side. In addition, the manipulating wire 20 will be described below in detail.

As illustrated in FIGS. 2A and 2B, in the clipping treatment device 10 of the embodiment of the invention, for example, the three clips 12 (12A, 12B, and 12C) are connected together, and are accommodated in the sheath 16 as the clip series A. The repetitive clipping treatment device 10 of this embodiment is a three-barreled clipping treatment device 10 which can perform clipping three times continuously, without withdrawing the sheath 16 from the interior of a patient's body.

In addition, the clipping treatment device 10 is not limited to the clipping treatment device loaded with three clips 12, and two clips may be loaded or four or more clips may be loaded.

The clips 12 are respectively connected together by connection rings 14 (14A, 14B, and 14C) which covers an engagement portion between the preceding and subsequent clips 12 and maintains a connected state between the clips 12. Additionally, the dummy clip 18 which is engaged with the manipulating wire 20 is connected to the clip 12C nearest to the proximal end side.

Although described in detail below, the manipulating wire 20 is inserted through the interior of the sheath 16, is inserted through the manipulation handle 48 which will be described later, and is connected to the slider 62 which performs preparation of clipping and manipulation.

In this embodiment, the forward end side is also referred to as “preceding”, and the proximal end side opposite to a part on the forward end side is also referred to as “subsequent” or “next”. Additionally, the clip immediately before a given clip 12 is also referred to as the “previous clip 12”, and similarly, the clip 12 immediately after the certain clip is also referred to as the “next clip 12” or a “subsequent clip 12”.

FIGS. 2A and 2B show the initial state immediately before a clipping treatment operation by the foremost clip 12A (standby state) is started.

As illustrated in FIGS. 2A and 2B, one clip 12 and one connection ring 14 including one clamping ring 40 which will be described below constitute one bleeding stopping clip body B for an endoscope (hereinafter referred to as a clip body B). In the form of the clip body B, plural clips 12 is loaded into the interior of the forward end of the long sheath 16.

In addition, although FIGS. 2A and 2B illustrate a state where the first foremost clip 12A protrudes from the forward end of the sheath 16, when the three clips 12A to 12C are loaded into the sheath 16, the first foremost clip 12A is completely housed inside the sheath 16.

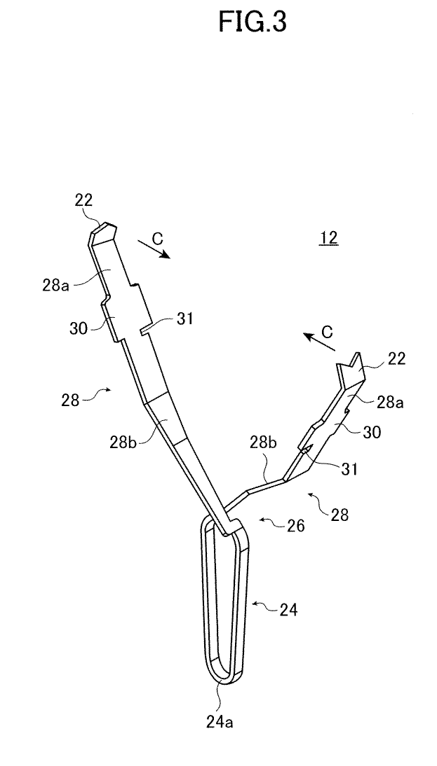

FIG. 3 is a perspective view illustrating the schematic configuration of a clip used for the clipping treatment device illustrated in FIG. 1.

The clip 12 is obtained by curving a single elongated plate having a resilience by 180° to form a closed end, and then crossing both the pieces thereof to form claw portions 22 and 22 that are bent in directions in which two open ends face each other. With the crossing portion 26 as a border, arm portions 28 and 28 are formed on the open end side, and a turned portion 24 is formed on the closed end side. The arm portions 28 and 28 are respectively provided at predetermined angles at opening-side ends of the turned portion 24 so that the arm portions cross each other at the crossing portion 26 and the distance therebetween becomes gradually larger. For example, the turned portion 24 is obtained by molding one member in a substantial U shape, and a tail portion 24a is rounded. The turned portion 24 is open, for example, in a direction orthogonal to a closing direction C of the arm portion 28.

Partially widened projections 30 and 30 are respectively formed at center portions of the arm portions 28 and 28, and each arm portion 28 is divided by the projection 30 into a distal portion 28a on the claw portion 22 side, and a proximal portion 28b on the crossing portion 26 side.

In the arm portions 28 and 28, cutouts 31 for reliably fitting the clamping ring 40 are respectively formed on the crossing portion 26 side of the projections 30 and 30. The clamping ring 40 fixed to the forward end portion of the connection ring 14 fitted to the crossing portion 26 of the clip 12 moves by a predetermined amount toward the claw portions 22 and 22, that is, toward the projections 30 and 30 while pressing the proximal portions 28b and 28b of the arm portions 28 and 28 in mutually facing directions, whereby the arm portions 28 and 28 and the claw portions 22 and 22 are closed, and the claw portions 22 and 22 exert a predetermined cusp-fitting force, that is, a gripping force.

In order to reliably pinch target portions, such as a bleeding portion or a portion to be treated after the removal of a diseased tissue, the claw portions 22 and 22 are formed into V-shaped male type and female type which are engaged with each other.

Additionally, as illustrated in FIG. 3, the width of the distal portion 28a of the arm portion 28 of the clip 12 remain constant and invariable from the claw portion 22 to the projection 30, while the width of the proximal portion 28b thereof gradually increase from the crossing portion 26 toward the projection 30 and is constant in the vicinity of the projection 30. This facilitates and ensures the movement of the clamping ring 40, thereby facilitating and ensuring the opening, closing, and fitting of the claw portion 22 and 22, and facilitating and ensuring the stopping of bleeding, or the suturing or closure of a wound, and the like in a living body or the like.

In addition, a biocompatible metal is preferably used for the clip 12. For example, it is possible to use JIS SUS630 or JIS SUS631, which is a precipitation-hardened stainless steel.

As for the clip 12, the arm portions 28 are retracted into the connection ring 14 which will be described below, whereby the space between the arm portions 28 becomes narrow, and living body tissue is clipped by the claw portions 22.

Here, the projections 30 have a width larger than the inner diameter of a forward-end-side opening of the connection ring 14 which will be described below, that is, a hole 41 of the clamping ring 40 and a proximal-end-side opening, that is, a hole 43 of a retaining portion 42. As a result, in the clipping treatment device 10, the portions of the clip 12 other than the projections 30 can enter the interior of the connection ring 14 due to the clip 12, but the projections 30 cannot enter the interior either from the forward end side or proximal end side of the connection ring 14.

In this embodiment, as illustrated in FIGS. 2A and 2B, the claw portions 22 are engaged with the turned portion 24 of the first clip 12A with the arm portions 28 of the second clip 12B closed, and the engagement portion is covered with and retained by the connection ring 14A, whereby the first clip 12A and the second clip 12B are brought into a connected state.

As illustrated in FIG. 2A, the claw portions 22 and 22 of the second clip 12B orthogonally mesh with the turned portion 24 of the first clip 12A so as to be combined therewith, and the first clip 12A and the second clip 12B are connected together in orientations differing by 90 degrees. Hereinafter, the third clip 12C is also connected to the second clip 12B in an orientation differing by 90 degrees. In the clip series A, the clips 12A to 12C are connected together with their orientations alternately changed every 90°.

The connection ring 14 acts as a clamping member of the arm portions 28 when clipping is performed, and is inserted so as to be able to advance or retreat in the longitudinal direction of the sheath 16 with the clips 12 housed. That is, the outer diameter of the connection rings 14 is substantially equal to the inner diameter of the sheath 16 so that the connection rings can smoothly advance or retreat and move within the sheath 16 as the clips 12 move.

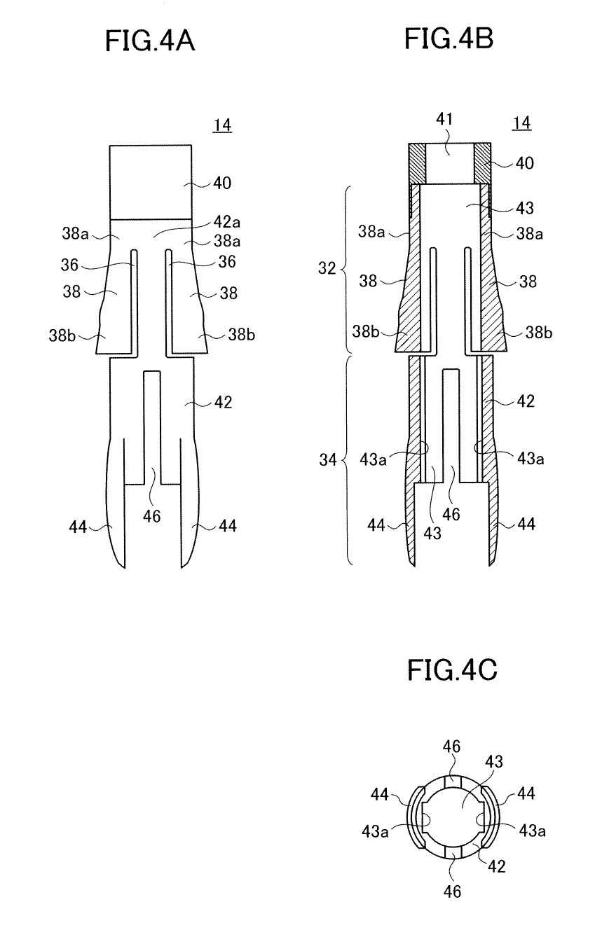

FIG. 4A is a front view illustrating an example of a connection ring used together with a clip of the repetitive clipping treatment device of the embodiment of the invention, FIG. 4B is a cross-sectional view of the connection ring illustrated in FIG. 4A, and FIG. 4C is a bottom view of the connection ring illustrated in FIG. 4A.

As illustrated in FIGS. 4A and 4B, the connection ring 14 has the clamping ring 40, and the retaining portion 42. In the connection ring 14, the clamping ring 40 made of metal is fastened to the forward end of the retaining portion 42 made of resin, and these two members are constructed integrally.

The retaining portion 42 formed of a resin serves to maintain the connected state of the clip 12 and to retain the clip 12 within the connection ring 14, and the clamping portion 40 formed of metal serves to clamp the clip 12. The connection ring 14 may be formed as a single member as long as the connection ring can exhibit the functions of both the clamping ring 40 and the retaining portion 42.

The clamping ring 40 is a cylindrical or ring-shaped metal part attached to the forward end side of the connection ring 14, and has the hole 41 whose inner diameter is larger than the width of the clip 12 in the vicinity of the crossing portion 26, and smaller than the width of each of the projections 30. Accordingly, although the clamping ring 40 can move in the vicinity of the crossing portion 26 of the clip 12 to be retained, the clamping ring does not escape toward the forward end side beyond the projections 30. That is, the projections 30 function as a stopper for determining the movement limit of the connection ring 14 that advances with respect to the clip 12.

The clamping ring 40 is set at a predetermined position in the vicinity of the crossing portion 26 of the clip 12. The clamping ring 40 moves from the initial position thereof, that is, from the crossing portion 26 toward the projections 30, with the arm portions 28 of the clip 12 increasing in width, so as to perform a clamping function of closing both the diverged arm portions 28 and 28 of the clip 12, and fixing the arm portions. A biocompatible metal is used for the clamping ring 40. By forming the clamping portion 40 of metal, a frictional force serving as a clamping force can be exerted on the metal clip 12.

The retaining portion 42 is a substantially cylindrical (ring-shaped) part which is resin-molded. The retaining portion 42 has a first region 32 which retains the preceding clip 12 and a second region 34 which is a connection retaining region that retains the next clip 12 while connected to the preceding clip. The retaining portion 42 has the hole 43 which communicates with the hole 41 of the clamping ring 40, and passes through the first region 32 and the second region 34.

The circular hole 43 is a circular hole which can accommodate the turned portion 24 of the clip 12, and the proximal portions 28b and 28b of the arm portions 28 and 28 thereof, and which is larger than the hole 41 of the clamping ring 40.

The peripheral surface of the forward end portion of the first region 32 is provided with a stepped portion for allowing the clamping ring 40 to be fitted thereto. The clamping ring 40 and the retaining portion 42 are fitted to each other by such interference fit as to prevent disengagement therebetween in a state where the clamping ring and the retaining portion are loaded into the sheath 16 and during clipping manipulation.

The first region 32 has a skirt portion 38 which inclines and diverges in the shape of a skirt with respect to the axis of the main body (not shown) of the connection ring 14.

In the skirt portion 38, the forward end side, that is, an upper base portion 38a in FIGS. 4A and 4B is connected to a main body 42a of the retaining portion 42, and a lower diverging portion 38b is partially disconnected from the main body 42a by a cut 36 formed in the main body 42a so as to be radially diverged or closed. Two skirt portions 38 are formed at two locations on both sides which are separated from each other by 180 degrees at the same position in the pulling direction of the clips 12, that is, in the vertical direction of FIGS. 4A and 4B.

As illustrated in FIG. 4A, the diverging portions 38b of both the skirt portions 38 and 38 are diverged in the shape of a skirt in a natural state where no external force is imparted thereto. At this time, the interior of the first region 32 of the retaining portion 42 forms a columnar space, as illustrated in FIG. 4B.

On the other hand, when the connection ring 14 is loaded into the sheath 16, as in the case with, for example, the second connection ring 14B illustrated in FIG. 2B, the diverging portions 38b of the skirt portions 38 are pressed against the inner surface of the sheath 16 and are inwardly pushed to enter the internal space, and the inner-circumferential-side portions of the diverging portions 38b of the skirt portions 38 press the side surface of the turned portion 24 of the second clip 12B retained by the first region 32 to retain and keep the second clip 12B from moving in a rotating direction and an advancing/retreating direction within the connection ring 14B. The skirt portions 38 may press and retain the clip retained by the second region 34, that is, the subsequent clip.

As in the case with the first connection ring 14A illustrated in FIG. 2A, on coming out from the forward end of the sheath 16, the skirt portions 38 and 38 are opened due to the elasticity of the skirt portions 38 and 38 themselves, thus releasing the first clip 12A from retention, and becoming wider than the inner diameter of the sheath 16 to prevent the connection ring 14A from retreating into the sheath 16. In this state, the manipulating wire 20 is pulled, and the first clip 12A retreats, whereby the connection ring 14A advances relative to the first clip 12A to clamp the first clip 12A by the clamping ring 40 of the forward end portion of the connection ring 14A.

Accordingly, it is necessary for the skirt portions 38 to have elasticity so as to be capable of being inwardly closed within the sheath 16, and diverged in the shape of a skirt when the skirt portions get out of the forward end of the sheath 16 and released from an external force. At the same time, it is also necessary for the skirt portions 38 to have rigidity which enables the clip 12 to be retained within the sheath 16, and rigidity which withstands the repulsive force of the clamping force of the clip 12 at the forward end of the sheath 16.

From those viewpoints, a material having biocompatibility and providing elasticity and rigidity required for the skirt portions 38 is used for the retaining portion 42. In addition, the shape of the retaining portion is determined so as to provide elasticity and rigidity required for the skirt portions 38. Examples of a material that can be used for the retaining portion 42 include polyphenylsulfone (PPSU), aromatic nylon, and the like. For ease of production, the retaining portion 42 is preferably molded integrally.

The second region 34 is provided on the proximal end side of the first region 32. The second region 34 retains the next clip 12 engaged with the clip 12 retained by the first region 32, specifically, the claw portions 22 and 22 and the distal portions 28a and 28a of the arm portions 28 and 28 in a state where the claw portions 22 and 22 are closed with the closed end, that is, tail portion 24a of the turned portion 24 of the preceding clip 12 being pinched therebetween.

The second region 34 has a length which is substantially equal to the movement distance required for the clamping ring 40 set at the initial position with respect to the clip 12 to move until completing the clamping of the clip 12 as a region length. That is, while the clip 12 retreats relative to the connection ring 14 so as to be clamped, the second region 34 of the connection ring 14 maintains the connection between the two clips 12 and 12 retained therein so as to allow the pulling force of the subsequent clip 12 to be transmitted to the preceding clip 12, and when the clamping by the clamping ring 40 has been completed, the engagement portion between the two clips 12 and 12 is detached from the second region 34, thereby releasing the connection between the clips 12 and 12.

The second region 34, as illustrated in FIG. 4B, is formed with a hole 43 having the same inner diameter, which extend from the first region 32.

As illustrated in FIG. 4C, two grooves (recesses) 43a are formed in the inner surface of the hole 43 of the retaining portion 42 in the second region 34 so as to be opposed to each other. The grooves 43a are formed on the same side as the skirt portions 38.

Additionally, as illustrated in FIGS. 4B and 4C, slits 46 cut from the proximal end of the second region are formed at two opposed positions which rotate by 90° from the opposed direction of the grooves 43a in the second region 34.

In the retaining portion 42, an extension portion 44 is provided at a position where each groove 43a is provided so that a portion of the retaining portion on the proximal end side extends. That is, two extension portions 44 are provided so as to be opposed to each other similarly to the grooves 43a. The extension portion 44 is constituted by a plate-shaped member which is curved along the outer periphery of the retaining portion 42.

The grooves 43a and 43a can accommodate therein the distal portions 28a and 28a of the arm portions 28 and 28 of the clip 12 retained by the second region 34, with the claw portions 22 and 22 being closed.

Plate surfaces of the distal portions 28a and 28a of the arm portions 28 and 28 of the clip 12 retained by the second region 34 come in contact with inner walls of the grooves 43a and 43a. Additionally, the width of the groove 43a is smaller than the width of each of the projections 30 formed in the arm portions 28.

Accordingly, the projections 30 of the clip 12 retained by the second region 34 cannot enter the grooves 43a.

Additionally, the extension portions 44 and 44 are formed so as to extend from the grooves 43a and 43a, and plate surfaces of the distal portions 28a and 28a of the arm portions 28 and 28 of the clip 12 also come in contact with inner walls of the extension portions 44 and 44, subsequently to the inner walls of the grooves 43a and 43a. Even if the manipulating wire 20 is pulled during clipping treatment and the claw portions 22 and 22 of the posterior clip 12 reach the extension portions 44 and 44 from the grooves 43a and 43a, the claw portions 22 and 22 of the anterior clip 12 gripping the turned portion 24 of the posterior clip 12 are retained in a closed state. By providing the extension portions 44 and 44 in this way, coming-off of the clips 12 at the forward end 16a of the sheath 16 can be reliably prevented.

This can prevent, for example, the clip 120 which is the posterior clip 12 from retraction into, for example, the clip 12B which is the anterior clip 12. As a result, it is possible to maintain the relative positions of the anterior and posterior (preceding and subsequent) clips 12, and maintain the manipulation of pushing out the clip 12 by the manipulating wire 20 (refer to FIG. 5).

The engagement portion between the two clips 12 and 12 is located in the region of the second region 34 close to the boundary between the second region 34 and the first region 32. Since the turned portion 24 of the preceding clip 12, for example, the second clip 12B in the connection ring 14B of FIG. 2B is retained inside the sheath 16 by the closed skirt portions 38 in the first region 32, the advancing/retreating movement and the rotating movement of the clip are restrained. Additionally, the next clip 12, for example, the third clip 12C in the connection ring 14B of FIG. 2B which is engaged with the preceding clip 12 is retained by the grooves 43a of the second region 34 in a direction different from the preceding clip by 90°, whereby the rotating movement thereof is restrained. The next clip 12 is also engaged with the preceding clip 12 whose advancing/retreating movement has been restrained, whereby the advancing/retreating movement thereof is restrained. That is, the engagement portion between the preceding and subsequent clips is retained by the connection ring 14 with very little play.

The slits 46 are formed up to positions shallower than the upper end of the second region 34. In other words, the slits 46 are provided at positions 90-degree shifted from the diverging direction of the clips 12 retained by the second region 34.

By providing the slits 46, the flexibility of the connection ring 14 can be improved, and the sheath 16 of the clipping treatment device 10 can pass through a curved portion with small curvature. Additionally, by providing the slits 46, the extension portions 44 and 44 provided at a skirt (proximal end portion) of the connection ring 14 can be expanded. Therefore, when the preceding and subsequent clips 12 and 12 are connected together prior to the loading of the clips 12 into the sheath 16, easy connection can be made advantageously by expanding the skirt and the extension portions 44 and 44 of the connection ring 14.

The extension portions 44 and 44 are located outside the grooves 43a and 43a, and the arm portions 28 and 28 of the posterior clip 12 are retained, subsequently to the grooves 43a and 43a. Additionally, since the clip body B is retained so as not to fall from the forward end of the sheath 16, the outer diameter of the extension portions 44 and 44 is smaller than the inner diameter of the sheath 16, and is larger than the retaining portion 42.

Moreover, since the projections 30 of the clip 12 and the extension portion 44 of the connection ring 12 comes into contact with each other when the clip 12 is rotated in order to change the diverging direction (the closing direction C) of the clip 12, it is possible to transmit a force in the rotating direction to the clip 12 at a higher efficiency.

As illustrated in FIGS. 2A and 2B, the rearmost third clip 12C is engaged with the dummy clip 18, which is not used for clipping treatment. The dummy clip 18 has, at the forward end portion thereof, a resilient portion having a shape similar to that of the open-end-side half portion of the clip 12 extending from the crossing portion 26. The resilient portion is engaged with the turned portion of the third clip 12C, with the claw portions thereof being closed, and releases the third clip 12C when the claw portions are opened. Additionally, the connecting member 19 is attached to the proximal end portion of the dummy clip 18. The connecting member 19 is detachably connected to the connection member 21 at the forward end of the manipulating wire 20 which will be described below.

The manipulating wire 20 moves the clip series A to the forward ends side of the sheath 16 or the proximal end side of the sheath 16 in the longitudinal direction of the sheath 16 inside the sheath 16 in clipping treatment 10. That is, the manipulating wire 20 advances or retreats the clip series A in the longitudinal direction thereof inside the sheath 16. The manipulating wire 20 is formed from, for example, a metal wire.

The manipulating wire 20 is housed in the sheath 16, and the connection member 21 is provided at the forward end, that is, at the edge opposite to the manipulating portion 50 as described above as described above.

The forward end portion of the manipulating wire 20 is connected by the connection member 21 to the clip 12 via the connecting member 19 and the dummy clip 18, while the proximal end portion thereof to which the connection member 21 is not attached is connected to the manipulating portion 50. Additionally, as described above, the proximal end portion 16c of the sheath 16 is also attached, together with the manipulating wire 20, to the manipulating portion 50 which will be described below.

The connection member 21 is obtained, for example, by connecting two substantially conical members with their bottom surfaces being opposed to each other, and is constituted by an elastic member, such as rubber. The connecting member 19 is formed with a recess having the same shape as this connection member 21. The connection member 21 of the manipulating wire 20 is fitted into the connecting member 19 of the dummy clip 18. As a result, even when the manipulating wire 20 is pulled to the proximal end side and even when the manipulating wire is pushed out to the forward end side, the bottom surface of any conical member of the connection member 21 is caught. Therefore, the connection member 21 can be prevented from being detached from the connecting member 19, thereby advancing or retreating the clips 12 in the longitudinal direction of the sheath 16.

Next, the configuration of the manipulation handle 48 of the clipping treatment device 10 of the embodiment of the invention will be described.

FIG. 5 is a schematic cross-sectional view illustrating a handle portion of the manipulation handle of the repetitive clipping treatment device of the embodiment of the invention.

In this embodiment, the manipulation handle 48, as described above, has the sheath 16, the manipulating wire 20, the connecting member 21 at the forward end of the manipulating wire 20, and the manipulating portion 50.

As illustrated in FIG. 5, the manipulating portion (handle portion) 50 of the manipulation handle 48 has a main body rail 52 which constitutes a handle main body, a forward end member 54 which is fixed to the forward end of the main body rail 52, a finger hook ring 56 which is attached to the proximal end of the main body rail 52, and a wire fixing member 58 which is provided outside the main body rail 52, an adjustment dial 60, a slider 62, a lock dial 64, a slider guide (slider displacement regulating member) 66 which is provided outside the forward end member 54, and a position regulating member 68.

In addition, in this embodiment, the longitudinal direction of the main body rail 52 is referred to as the “axial direction”, and the circumferential direction around this axial direction is referred to as the “circumferential direction”.

The proximal end portion 16c of the sheath 16 is retained by the forward end of the forward end member 54, and a proximal end 20b of the manipulating wire 20 is fixed so as to coincide with a wire connecting portion 58a of the wire fixing member 58 (the central axis of the manipulating portion 50). For example, an operator can pass his/her thumb through a ring portion 56a of the finger hook ring 56, hook his/her index finger and middle finger on the slider 62, and slidingly move the slider 62 in the advancing/retreating direction with respect to the finger hook ring 56. When the slider 62 moves, the wire fixing member 58 also moves, and the manipulating wire 20 retained by the wire connecting portion 58a also moves. On the other hand, the sheath 16 is connected to the finger hook ring 56 via the forward end member 54 and the main body rail 52. Accordingly, the manipulating wire 20 can be can be moved to advance or retreat the sheath 16 by the movement manipulation of the slider 62 with respect to the finger hook ring 56.

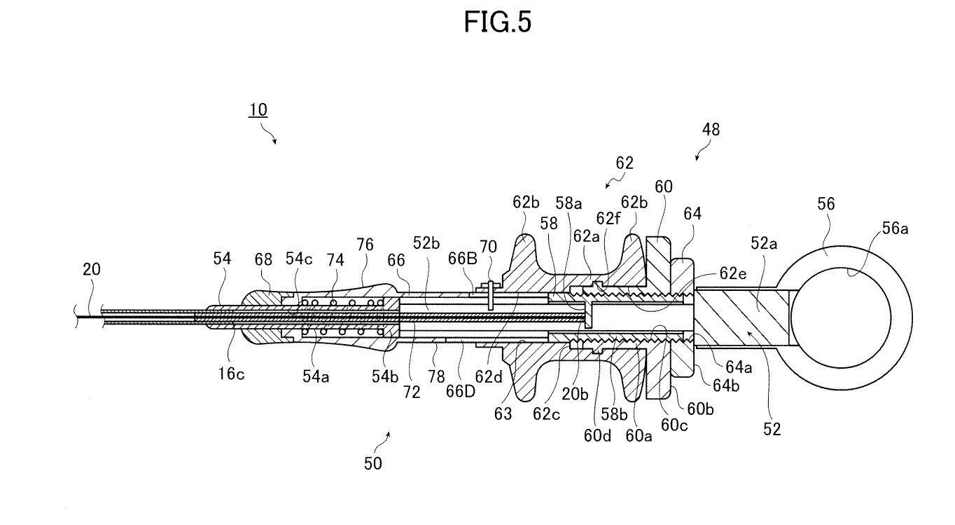

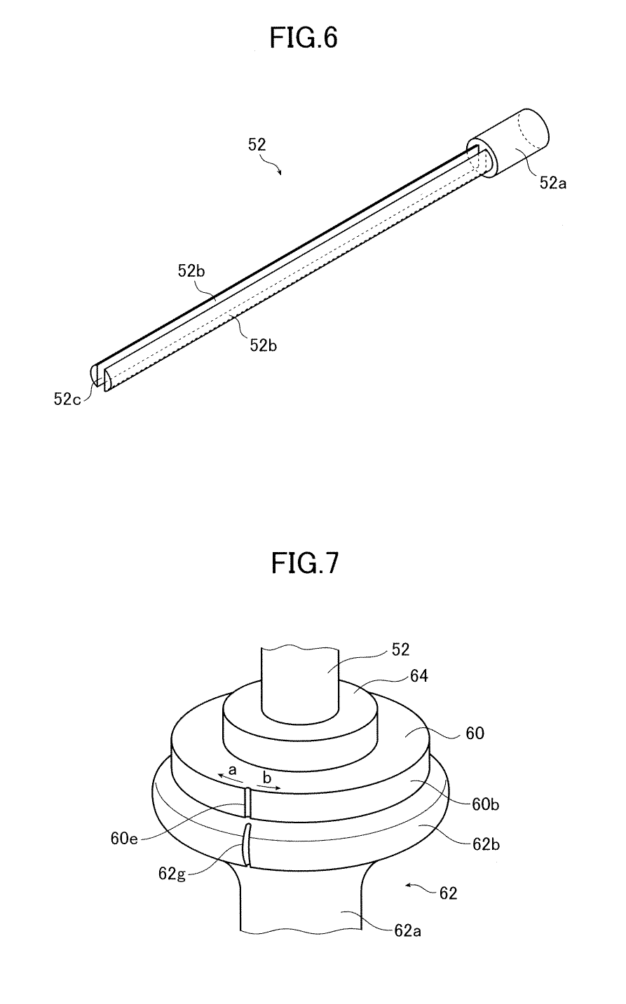

In the main body rail 52, as illustrated in FIG. 6, two rod members 52b having a substantially semi-circular cross-section are provided at a columnar proximal end portion 52a so that their planar portions are opposed to each other and have a gap 52c with a predetermined interval therebetween. For example, the respective rod members 52b and 52b are formed by shaving off the center of a cylinder that is coaxial with and thinner than the proximal end portion 52a, by a predetermined width along the axis. In FIG. 5, the rod member 52b of the main body rail 52, on the near side of the drawing is removed, and the planar portion of the rod member 52b on the far side is shown.

The gap 52c between the two rod members 52b and 52b is attached to the wire connecting portion 58a of the wire fixing member 58, and the slider 62, and is set to a dimension such that a forward end portion of a slider pin 70 extending toward the center of the main body rail 52 can be inserted into the gap. As illustrated in FIG. 5, the forward ends of the wire connecting portion 58a and the slider pin 70 can be inserted into the gap 52c between the two rod members 52b and 52b, and guided by the rod members 52b and 52b, moved in the extension direction of the main body rail 52. That is, the rod members 52b and 52b of the main body rail 52 function as rails for the wire connecting portion 58a (wire fixing member 58) and the slider pin 70 (slider 62).

The forward end member 54 has an elongated tubular portion 54a and a flange portion 54b with a large diameter provided at the edge of the tubular portion 54a, and is formed with a through hole 54c which passes through the tubular portion 54a and the flange portion 54b. The forward end of the main body rail 52 is fixed to the proximal end surface of the flange portion 54b. Additionally, the proximal end portion 16c of the sheath 16 is inserted into and retained by the forward end of the tubular portion 54a. The manipulating wire 20 extends from the proximal end of the sheath 16, and passes through the through hole 54c of the forward end member 54. The outer diameter of the tubular portion 54a of the forward end member 54 is approximately equal to the inner diameter of the forward end portion (grasping portion 76) of the slider guide 66 arranged outside the forward end member 54, and the outer diameter of the flange portion 54b is approximately equal to the inner diameter of a rear end portion (guide portion 78) of the slider guide 66. As a result, the slider guide 66 is allowed to slide in the circumferential direction with respect to the forward end member 54.

The finger hook ring 56 has a ring portion 56a into which an operator's finger can be inserted, and is attached to the cylindrical proximal end portion 52a of the main body rail 52.

The wire fixing member 58 is a cylindrical member which has an inner diameter approximately equal to the outer diameter of the two rod members 52b and 52b of the main body rail 52. The outer peripheral surface of the proximal end portion of the wire fixing member 58 is formed with a male thread 58b. The wire connecting portion 58a which protrudes to the inner surface side is formed substantially at an axial central portion of the wire fixing member 58. The proximal end 20b of the manipulating wire 20 which extends to the proximal end side (rearward) through the forward end member 54 is fixed to the position of the wire connecting portion 58a which coincides with the central axis (not illustrated) of the wire fixing member 58. In addition, the manipulating wire 20 is covered with a reinforcing pipe 72 in a range from a portion which has slightly entered the forward end side (front side) from the proximal end portion 16c of the sheath 16, to the wire connecting portion 58a, is reinforced so as to maintain a substantially linear shape, without being bent or curved inside the manipulating portion 50.

The adjustment dial 60 is a cylindrical part with a flange which is fitted to the outside of the wire fixing member 58, and has a cylindrical portion 60a and a flange portion 60b provided at the proximal end thereof. The inner peripheral surface of the cylindrical portion 60a of the adjustment dial 60 is formed with a female thread 60c, and the female thread 60c is screwed to the male thread 58b of the wire fixing member 58. The flange portion 60b is rotated by the operator, whereby the adjustment dial 60 advances or retreats together with the male thread 58b of the wire fixing member 58 according to the rotating direction of the flange portion. An annular engagement projection 60d is formed in the vicinity of the forward end of the outer peripheral surface of the cylindrical portion 60a of the adjustment dial 60 over its entire circumference. The engagement projection 60d is engaged with an annular engagement recess 62f over the entire circumference of the inner peripheral surface of the slider 62 which will be described below. Additionally, as illustrated in FIG. 7, the home position mark 60e is provided in the adjustment dial 60.

The home position mark 60e is formed in the shape of a groove along a generatrix in the outer peripheral surface of the flange portion 60b of the adjustment dial 60. The home position mark 60e is aligned with a home position mark 62g provided in the shape of a notch in the outer peripheral surface of a larger-diameter portions 62b of the slider 62 described next, and thereby positions the wire fixing member 58 at an axial predetermined position with respect to the slider 62. That is, the home position mark 60e on the adjustment dial 60 side is caused to coincide with the home position mark 62g on the slider 62 side, whereby the position of the proximal end 20b of the manipulating wire 20 is regulated to a predetermined position (home position) with respect to the slider 62, and eventually the main body rail 52.

Here, the adjustment dial 60 in a state where the home position mark 60e is aligned with the home position mark 62g on the slider 62 side is rotated in one direction, for example, in the direction of an arrow a of FIG. 7, whereby the wire fixing member 58 is moved to the proximal end side, that is, to the rear side (the right side in FIG. 5) to pull the manipulating wire 20 to the proximal end side, and rotating the manipulating wire reversely in the other direction, for example, in the direction of an arrow b in FIG. 7), so that the wire fixing member 58 can be moved to the forward end, that is, the front, side (the left side in FIG. 5) to push out the manipulating wire 20. That is, the manipulating wire 20 can be pulled or pushed by rotating the adjustment dial 60 with the home position mark 60e being aligned, in one direction or in the other direction. In addition, the pulling-side stroke and pushing-side stroke of the manipulating wire 20 are not necessarily caused to coincide with each other. For example, when fine adjustment on the pulling side of the manipulating wire 20 with respect to the sheath 16 is large, the pulling-side stroke may be enlarged.

The slider 62 is mounted so as to be axially movable on the outer periphery of the main body rail 52 which constitutes the handle main body, and has a cylindrical portion 62a, and larger-diameter portions 62b formed at both ends of the cylindrical portion 62a.

Additionally, the slider 62 is formed with a hole 63 which passes through the cylindrical portion 62a and each of the larger-diameter portions. The slider 62 is formed in the shape of a bobbin as a whole.

In the manipulating portion 50 of this embodiment, the operator inserts their thumb into the ring portion 56a of the finger hook ring 56, pinches the cylindrical portion 62a lightly with his/her index finger and middle finger, and pushes the proximal surface of the front larger-diameter portions 62b with the backs of his/her index finger and middle finger, so that the slider 62 can be moved to the forward end side. On the contrary, the operator pulls the forward-end-side surface of the rear larger-diameter portions 62b to the rear end side with the insides of his/her index finger and middle finger, so that the slider 62 can be moved to the proximal end side.

On the other hand, in the slider 62, an enlarged diameter portion 62e is formed on the proximal end side of the hole 63. The annular engagement recess 62f is formed in the enlarged diameter portion 62e over its entire inner periphery.

The wire fixing member 58 and the adjustment dial 60 are inserted through the hole 63 of the slider 62 from the enlarged diameter portion 62e side.

The wire fixing member 58 is inserted into the hole 63 from the enlarged diameter portion 62e side, and the forward end thereof reaches a position exceeding the stepped portion 62c of the edge of the enlarged diameter portion 62e.

Additionally, the cylindrical portion 60a of the adjustment dial 60 is inserted into the enlarged diameter portion 62e, with the inner female thread 60c being screwed to the male thread 58b of the wire fixing member 58, and the engagement projection 60d of the outer peripheral surface of the cylindrical portion 60a being engaged with the engagement recess 62f of the enlarged diameter portion 62e. As a result, although the adjustment dial 60 moves integrally with a slider 62 in the axial direction, the adjustment dial can rotate freely with respect to the slider 62 in the circumferential direction.

The lock dial 64 is a ring-shaped part in the inner peripheral surface of which a female thread 64a to be screwed to the male thread 58b of the proximal end portion of the wire fixing member 58 is formed. After the position of the wire fixing member 58 is adjusted by the adjustment dial 60, the position of the adjustment dial 60 after the adjustment is fixed by fastening the lock dial 64 until the lock dial hits the proximal end surface of the adjustment dial 60.

The slider 62 and the adjustment dial 60 are engaged with each other by their respective engagement recess 62f and engagement projection 60d, and the adjustment dial 60 and the lock dial 64, and the wire fixing member 58 are screwed together by a threaded portion. For this reason, when the slider 62 is moved to the forward end side of the sheath 16 or the proximal end side of the sheath 16, these four parts move integrally.

In addition, the movement limit of the slider 62 on the proximal end side is regulated by a position where the proximal end surface 64b of the lock dial 64 comes into contact with the forward end surface of the cylindrical portion 52a of the main body rail 52

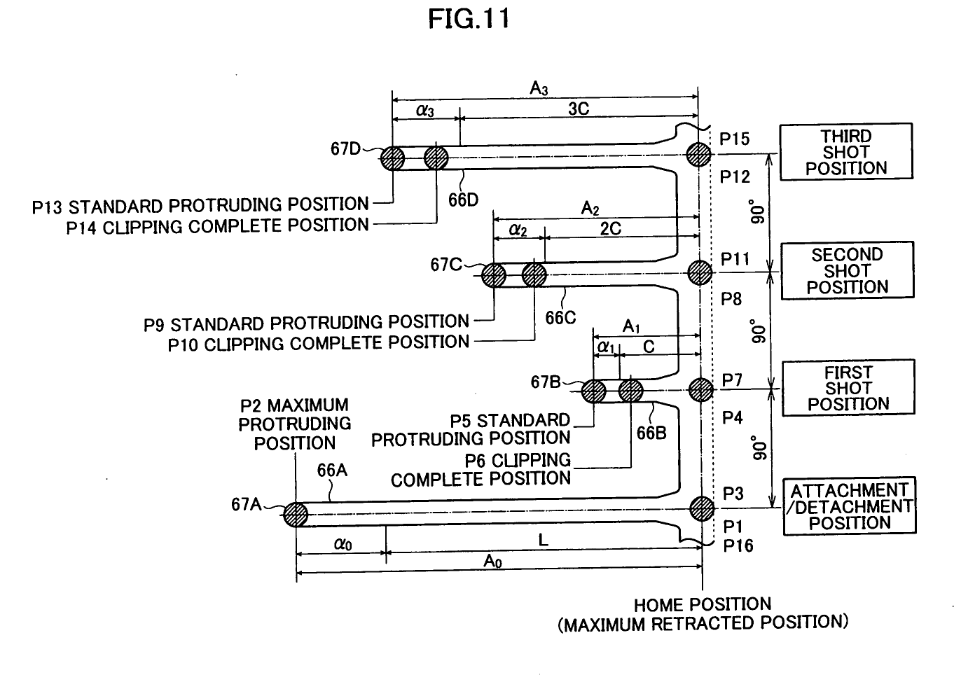

In the clipping treatment device 10, the slider 62 is moved to the proximal end side of the sheath 16, and the position where the proximal end surface 64b of the lock dial 64 comes into contact with the forward end surface of the cylindrical portion 52a of the main body rail 52 becomes a home position (hereinafter referred to as HP) of the slider 62. The position of the slider pin 70 at this time, for example, the position P1 of FIG. 11 which will be described below, is referred to as being at HP.

The slider pin 70 is fixed to the forward end portion of the slider 62 by being inserted into the inside from the outside. The forward end of the slider pin 70 extends toward the center of the main body rail 52, and arrives at the gap 52c between the respective rod members 52b of the main body rail 52. The slider pin 70 can move the gap 52c between the two rod members 52b in the front-back direction, that is, in the axial direction.

The slider guide 66 is mounted on the outer periphery of the main body rail 52 which constitutes the handle main body so as to be rotatable in the circumferential direction of the main body rail 52, and regulates the displacement of the slider 62 in the axial direction of the main body rail 52 to several different displacements which are required for the clipping treatment of each of the clips 12A to 12C of the clip series A. The slider guide 66 is constituted by a substantially cylindrical member provided outside the forward end member 54.

As described above, the inner diameter of the forward-end-side handle portion 76 of the slider guide 66 is approximately equal to the outer diameter of the tubular portion 54a of the forward end member 54, and the inner diameter of the proximal-end-side guide portion 78 of the slider guide 66 is set to be approximately equal to the outer diameter of the flange portion 54b of the forward end member 54, whereby the whole slider guide 66 is slidably supported by the flange portion 54b. Additionally, the outer diameter of the guide portion 78 of the slider guide 66 is slightly smaller than the inner diameter of the slider 62, so that when the slider 62 moves to the forward end side, the slider can enter the interior of the slider 62. Since the slider guide 66 is rotationally moved with respect to the position regulating member 68 at the forward end thereof by operator's manipulation, the outer surface of the slider guide may be formed with a slope (not illustrated) outside so that the operator easily carry the slider guide.

A coiled spring 74 is arranged around the forward end member 54 between the flange portion 54b of the forward end member 54, and the inner surface of the slider guide 66. The coiled spring 74 is a compression spring, and urges the slider guide 66 to the forward end side with respect to the tubular portion 54a of the forward end member 54 which is a fixing member, and presses the slider guide against the position regulating member 68. The position regulating member 68 is fixed to the forward end member 54 fixed to the main body rail 52. That is, the finger hook ring 56 is fixed to the proximal end side of the main body rail 52 around the main body rail 52, the forward end member 54 is fixed to the forward end side of the main body rail 52, and the position regulating member 68 and the sheath 16 are fixed to the forward end member 54. That is, the main body rail 52, the finger hook ring 56, the forward end member 54, the position regulating member 68, and the sheath 16 are constructed integrally.

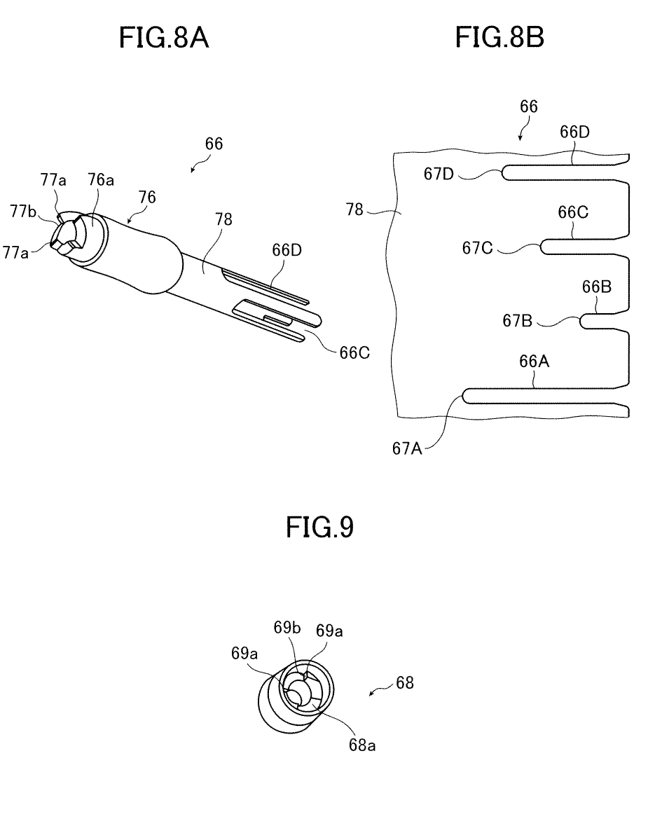

FIG. 8A is a schematic perspective view illustrating the slider guide of the manipulation handle of the repetitive clipping treatment device of the embodiment of the invention, and FIG. 8B is a schematic developed view illustrating the guide portion of the manipulation handle. FIG. 9 is a schematic perspective view illustrating a rotating position regulating member of the handle portion of the manipulation handle of the repetitive clipping treatment device of the embodiment of the invention.

As illustrated in FIG. 8A, the slider guide 66, the distal-end-side grasping portion 76 and the proximal-end-side guide portion 78 are formed integrally. A joint portion 76a which joins the position regulating member 68 is formed on the forward end side of the grasping portion 76, and four sawtooth-shaped projections 77a in which the inclination angles of two sides are different are formed at intervals of 90° in the circumferential direction at the end surface of the joint portion. In each sawtooth-shaped projection 77a, one surface becomes a gentle slope 77b whose inclination angle is gentle, and the inclination angle of the other surface is substantially a right angle. As illustrated in FIG. 9, four projections 69a and four gentle slopes (recesses) 69b therebetween are also provided in a joint portion 68a of the rear end portion of the position regulating member 68 which joins the slider guide 66, similarly to the joint portion 76a of the slider guide 66.

Since the slider guide 66 is pressed against the position regulating member 68 by the coiled spring 74 in a state where the slider guide meshes with the position regulating member 68, the slider guide 66 does not rotate with respect to the position regulating member 68 unless an external force is exerted thereon from the operator. Additionally, since the position regulating member 68 and the slider guide 66 mesh with each other by sawtooth-shaped irregularities, when the operator tries to turn the slider guide 66 around an axis, the slider guide 66 rotates in a direction in which mutual steep slopes (substantially right-angled surfaces) in the projections 77a of the joint portion 76a of and the projections 69a of the joint portion 68a of the position regulating member 68 separate from each other, and does not rotate in a direction in which the opposite steep slopes come into contact with each other. Although the slider guide 66 can rotate counterclockwise as seen from the proximal end side (the right side of FIG. 8A) in the illustrated example, the slide guide cannot rotate clockwise.

When the gentle slopes 77b of the joint portion 76a of the slider guide 66 rotate along the gentle slopes 69b of the joint portion 68a of the position regulating member 68, rotate by 90°, and rides over mutual peaks, the gentle slopes mesh with each other by the next irregularity. As a result, the slider guides 66 rotate at every 90°.

The guide portion 78 of the slider guide 66, as illustrated in FIG. 8B is formed with four slider guide grooves (position regulating grooves) 66A, 66B, 66C, and 66D which extend along the central axis of the main body rail 52 and extend linearly with different axial lengths in order to switch the displacement of the slider 62 to plural steps, that is, to regulate the displacement of the slider to two or more displacements. The four slider guide grooves 66A, 66B, 66C, and 66D are formed at predetermined intervals, for example, at intervals of 90° in the circumferential direction in the slider guide 66 so as to match the projections 77a of the grasping portion 77.

Additionally, the slider guide 66 is arranged in the positional relationship so that the guide groove 66A, the guide groove 66B, the guide groove 660, and the guide groove 66D overlaps the gap 52c of the main body rail 52 in order as the slider guide is rotated.

The slider guide grooves 66A to 66D have a function as a guide groove for the slider pin 70, and the width of the grooves is approximately equal to the diameter of the slider pin 70. Whenever the slider guide 66 rotates by 90°, any of the slider guide grooves 66A to 66D coincides with the rail position of the main body rail 52, that is, the position of the gap 52c between the two rod members 52b.

Additionally, the proximal ends of the four guide grooves 66A, 66B, 660, and 66D are chamfered in order to easily enter the respective grooves, without being caught in other portions of the slider pin 70 and the slider guide 66.

In this embodiment, when the slider guide groove 66A overlaps the gap 52c of the main body rail 52, the forward end portion of the slider pin 70 can be guided by the main body rail 52, and can be moved in the axial direction along the slider guide groove 66A. That is, the slider 62 can move in the axial direction along the slider guide groove 66A.

In addition, when any of the slider guide grooves 66B to 66D does not overlap the gap 52c of the main body rail 52, the slider pin 70 can move in the axial direction along the slider guide grooves 66B to 66D, that is, the slider 62 can move in the axial direction along the slider guide grooves 66B to 66D.

In this embodiment, the displacement of the slider 62 is regulated depending on the length of the slider guide grooves 66A to 66D. In other words, the displacement of the slider pin 70 is regulated by forward-end-side ends 67A to 67D of the respective slider guide grooves 66A to 66D of the sheath 16.

The positions of the forward-end-side ends 67A to 67D of the four guide grooves 66A, 66B, 66C, and 66D, as illustrated in FIG. 8B, respectively differ. Specifically, the forward-end-side end 67A of the guide groove 66A is formed on the foremost side of the sheath 16, and the positions of the forward ends 67D, 67C, and 67B are nearer the proximal end side, that is, are positions far from the sheath 16 and near the finger hook ring 56, in order of the guide groove 66D, the guide groove 66C, and the guide groove 66B.

As described above, the slider guide grooves 66A to 66D have different lengths, respectively. In this embodiment, the slider guide groove 66B is the shortest, and the slider guide groove 66C, the slider guide groove 66D, and the slider guide groove 66A becomes longer in this order.

The longest slider guide groove 66A and the shortest slider guide groove 66B are formed adjacent to each other.

The slider guide grooves 66C and 66D formed on the side where the slider guide groove 66A and the slider guide groove 66B are not adjacent to each other are formed in order from a slider guide groove having a shorter length, that is, in order of the slider guide grooves 66C and 66D, as they go in the circumferential direction directed to the slider guide groove 66B from the slider guide groove 66A on the non-adjacent side.

Additionally, in this embodiment, the longest slider guide groove 66A is used for the loading of the clip series A. The other slider guide grooves 66B to 66D are used for clipping treatment.

In addition, in the invention, the number of clips which can be loaded (repetitively shot) is not limited to three as mentioned above. Accordingly, when the number of clips 12 which can be located into the clipping treatment device (manipulation handle) is n, according to the number of clips n, the guide portion 78 of the slider guide 66 is formed with slider guide grooves of “n+1” obtained by adding 1 for the loading of the clip series A to this number n.

Additionally, since the slider pin 70, and the end surfaces of the slider guide grooves 66A to 66D come into contact with each other and exert a certain degree of force, it is preferable that both the slider pin and the slider guide groove end surfaces be made of materials having good slidability and wear resistance. By making the slider pin and the slider guide groove end surfaces of such materials, the wear of the slider pin and the slider guide end surfaces can be inhibited.

Additionally, it is also preferable to form a fluororesin or diamond-like carbon (DLC) film on the slider pin 70 and the end surfaces of the slider guide grooves 66A to 66D. Wear resistance and slidability can be improved by forming a film having the above composition. Particularly, the DLC film is formed to reduce friction resistance, that is, friction coefficient μ and increase surface hardness, so that wear resistance can be significantly improved compared to a case where any film is not formed on the slider pin and the slider guide grooves.

Here, the sheath 16 is, for example, about 2 m in length. As described above, it is known that, when the sheath 16 is curved after the manipulating wire 20 is caused to protrude by a predetermined amount from the forward end 16a of the sheath 16 with a state where the sheath 16 is extended straightly being a reference state, so-called retraction will occur where the manipulating wire 20 does not pass through the vicinity of the central axis of the sheath 16, and passes through the outside in the sheath 16 and that the forward end of the manipulating wire 20 is located inside the forward end 16a of the sheath 16.

When the retraction amount caused by this retraction is large, even if the clips 12 are moved to the forward end side with respect to the sheath 16 by a loading distance, the skirt portions 38 of the clip 12A cannot protrude completely from the forward end 16a of the sheath 16, and the clip 12A may not be brought into a state where a clipping treatment operation is possible. In addition, although the above-described retraction amount differs depending on the curved state of the sheath 16, the retraction amount may become about 6 mm.

Moreover, it is considered that this retraction amount is constant irrespective of the number of clips of the clip series A which are loaded if the curving of the sheath 16 is made the same. However, the inventor has further studied and discovered that the retraction amount differs depending on the position (loading distance) of loaded clips, that is, the number of remaining clips, in a clip series including plural clips.

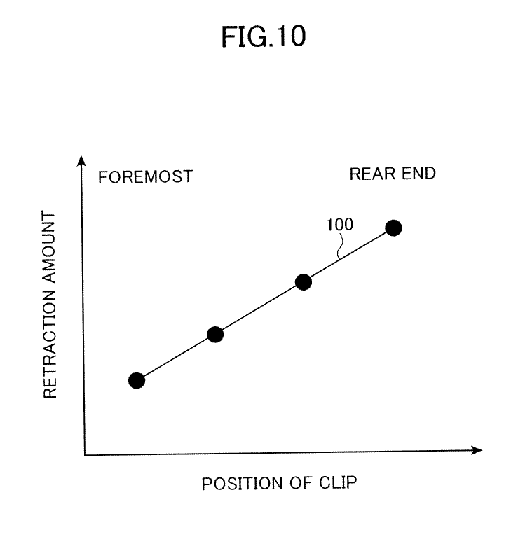

FIG. 10 shows a graph illustrating changes in the retraction amount depending on the position of a clip in a case where the number of clips is three. In addition, in FIG. 10, the foremost is a first shot clip and the rear end is a state where there is no clip. In FIG. 10, when the position of a clip of the horizontal axis increases, the number of remaining clips decreases.

As illustrated by a straight line 100 in FIG. 10, the retraction amount is smallest in a first shot clip, that is, in a state where three clips are present in the sheath 16. When clipping treatment is performed sequentially and the number of remaining clips decreases, that is, when the position of a clip changes to a second shot and a third shot, the retraction amount increases in proportion thereto. As such, the inventor has discovered that the number of remaining clips and the retraction amount in a clip series are in a proportional relationship.

For this reason, the difference in the retraction amount between a first shot clip and the last shot clip becomes larger as the number of clips in the clip series A increases. As a result, when the retraction amount is set by the first shot clip, in the third shot clip, the protruding of the connection rings 14 may be insufficient, and the skirt portions 38 may not open. On the contrary, when the retraction amount is set to the maximum, as for the first shot clip, the retraction amount is too large, and the clipping manipulation becomes unstable. Although the clip series A which has three clips has been described as an example in the invention, the possibility of the clipping treatment being hindered as described above as the number of clips increases to 4, 5, and 6 becomes high.

The reason why the retraction amount differs depending on the position of a clip of the clip series A illustrated in FIG. 10 is because the friction between the inner surface of the sheath 16 and a member, such as the connection rings 14 in the clip series A may change. For example, the skirt portions 38 press the inner surface of the sheath 16 within the sheath 16 due to the elasticity thereof, and this causes friction.

As the number of clips of the clip series A increases, the connection rings 14 increase, the friction caused by the skirt portions 38 increases, and the resistance against the tension to retract the manipulating wire 20 into the sheath 16, which is produced due to the difference in path length between the manipulating wire 20 and the sheath 16, becomes larger. For this reason, the retraction amount becomes small.

On the other hand, when the number of clips of the clip series A becomes small, the friction between the connection rings 14 and the inner surface of the sheath 16 decreases, and the resistance against the tension to retract the manipulating wire 20 into the sheath 16 also becomes small. For this reason, the retraction amount becomes large.

Particularly, since there is no resistance in a state which there is no clip, and only the connection member 21 is present in the manipulating wire 20, the retraction amount becomes the maximum.

Then, the inventor has discovered on the basis of the fact that the retraction amount changes depending on the position of a clip of the above-described clip series A and the number of remaining clips, since the amounts of retraction respectively differ in the respective clips 12A to clip 12C, the respective amounts of retraction are calculated in advance, and the respective amounts of retraction are included in the lengths of the respective slider guide grooves 66B to 66D. That is, the inventor has discovered that the respective slider guide grooves 66A to 66D are determined according to the number of clips of the clip series A on the basis of the respective amounts of retraction which are calculated in advance, and the length required to make a clip protrude from the forward end 16a of the sheath 16.

In this embodiment, as illustrated in FIG. 11, the length of the clip series A is defined as L, and the length of one clip is defined as C. In addition, the length C of this clip is a distance (loading distance) required to make the clips 12 protrude from the forward end 16a of the sheath 16. In the first shot clip, the length (loading distance) of the clip is C. In the second shot clip, the length (loading distance) of the clip is 2C. In the third shot clip, the length (loading distance) of the clip is 3C.

Additionally, the retraction amount in a state where there is no clip 12 is set to α₀, the retraction amount in a state where there are three clips 12A to 12C is set to α₁, the retraction amount in a state where there are two clips 12B and 12C is set to α₂, and the retraction amount in a state where there is one clip 12C is set to α₃.

In this embodiment, when the length of the slider guide groove 66A is set to A₀, A₀ is defined by A₀=α₀+L. Additionally, when the length of the slider guide groove 66B is set to A₁, A₁ is defined by A₁=α₁+C. Additionally, when the length of the slider guide groove 66C is set to A₂, A₂ is defined by A₂=α₂+2C. Additionally, when the length of the slider guide groove 66D is set to A₃, A₃ is defined by A₃=α₃+3C.

As such, the respective retraction amounts α₀ to α₃ according to the number of clips of the clip series A are calculated, the lengths of the slider guide grooves 66A to 66D are determined on the basis of the respective retraction amounts αₒ to α₃ and the lengths required to make the clips 12 protrude from the forward end 16a of the sheath 16, so that the respective clips 12A to 12C can be caused to protrude from the forward end 16a of the sheath 16, and the skirt portions 38 can be reliably opened. As a result, in the respective clips 12A to 12C, clipping treatment can be reliably performed.

Additionally, since the connection ring 14 is provided with the extension portions 44, even if the clips 12 are caused to protrude excessively, the clips 12 is prevented from coming off from the forward end 16a of the sheath 16.

In addition, the lengths of the respective slider guide grooves 66A to 66D, as illustrated in FIG. 11, are respectively distances from the position of the center of the slider pin 70 at the home position to the position of the center of the slider pin 70 in a state where the guide pin butts the forward ends 67A to 67D of the respective slider guide grooves 66A to 66D.

In this embodiment, in the slider guide groove 66A whose length is defined as described above, when the slider pin 70 is butted against the end 67A, for example, even when the sheath 16 is wound once and the forward end portion of the sheath is brought close to the proximal end portion, thereby making a large loop (one turn) or even if the sheath 16 is wound twice and two small loops are made (two turns), the connection member 21 provided at the forward end of the manipulating wire 20 protrudes from the forward end 16a of the sheath 16 by a predetermined length.

Additionally, even in the slider guide grooves 66B to 66D whose lengths are defined as described above, when the slider pin 70 is butted against the ends 67B to 67D, for example, an initial state (standby state) immediately before clipping treatment operations performed by the clips 12A to 12C are brought about. Here, the standby state means a usable state where the claw portions 22 and 22 can open to pinch a living body b or the like, like the clip 12A illustrated in FIGS. 2A and 2B.

In the above-described manipulation handle 48, when the sheath 16 is curved after the connection member 21 attached to the manipulating wire 20 is caused to protrude by a predetermined amount from the forward end 16a of the sheath 16 in a state where the sheath 16 is extended straightly, the forward end of the manipulating wire 20 will be retracted into the sheath 16 from the forward end 16a of the sheath 16 due to a difference in path length between the sheath 16 and the manipulating wire 20. At this time, when the length from the forward end 16a of the sheath 16 to the forward end of the manipulating wire 20 is set to the retraction amount α₀, this retraction amount α₀ will change variously according to the posture or curving direction of the sheath 16. For example, the retraction amount α₀ when the sheath 16 is wound twice and two small loops are made (two turns) tend to become larger than that when the sheath 16 is wound once and the forward end portion of the sheath is brought close to the proximal end portion, thereby making a large loop (one turn). For this reason, the retraction amount α₀ is set to the maximum.

Additionally the respective retraction amounts α₁ to α₃ are measured under the same conditions as, for example, the conditions under which the retraction amount α₀ is measured.

For this reason, it is necessary to set the posture of the sheath 16 so that the measuring conditions of the respective retraction amounts α₀ to α₃ become fixed. In this case, for example, the manipulation handle 48 is provided with an attachment portion (not illustrated) which attaches the sheath 16, the position of the sheath 16 with respect to this attachment portion is determined, for example, two turns are performed to attach the sheath 16, and this state is adopted as the measuring conditions.

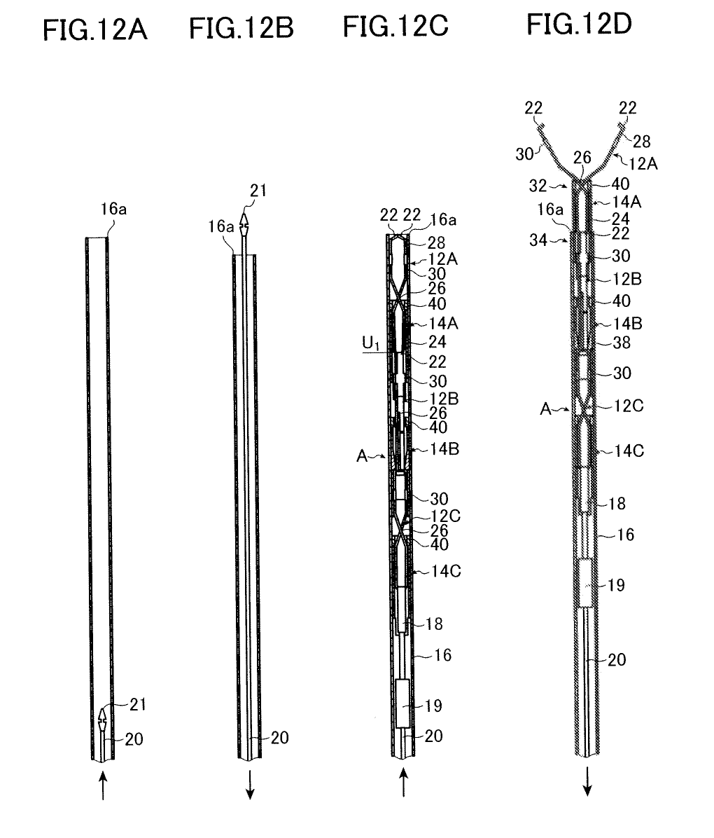

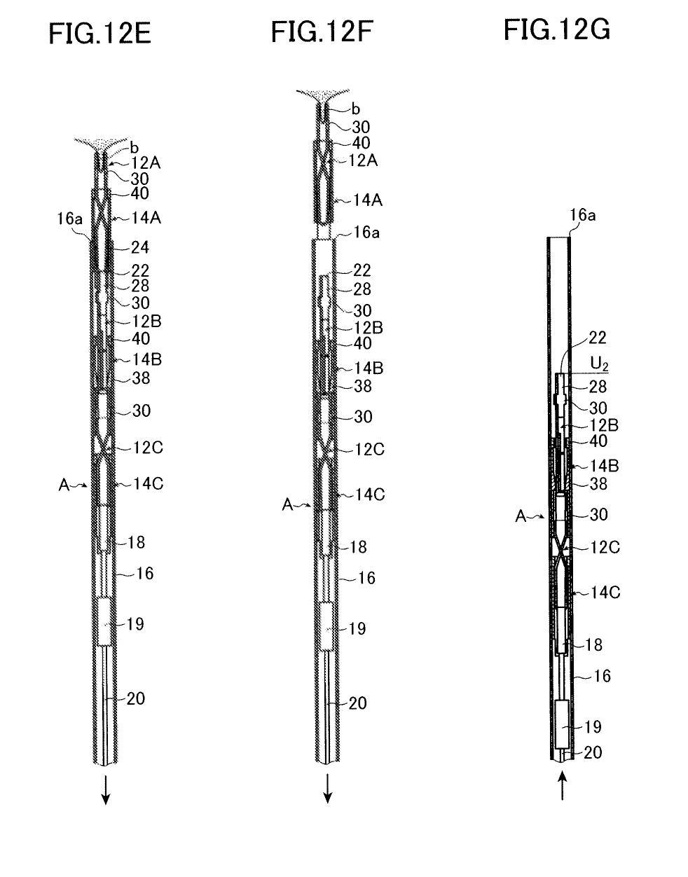

Next, the manipulation of clipping in the clipping treatment device 10 will be described with reference to FIG. 11 and FIGS. 12A to 12G.

FIGS. 12A to 12G are schematic cross-sectional views illustrating a gradual state in the clipping manipulation of the repetitive clipping treatment device of the embodiment of the invention.

In addition, the position of the slider pin 70 is indicated by circles in FIG. 11.

Additionally, in the description using FIG. 11, moving the slider pin 70 to the position 21 illustrated in FIG. 11 or the like is described as “moving the slider 62 to the position P1”, “moving the slider 62 to HP”, or the like while the slider pin 70 is omitted.

First, the slider guide 66 is rotated to set the slider 62 to the position P1 illustrated in FIG. 11 so as to be aligned with the slider guide groove 66A. At this time, the slider pin 70 is at the position P1, and as illustrated in FIG. 12A, the connection member 21 provided at the forward end of the manipulating wire 20 is located at a position where the connection member is withdrawn from the forward end 16a of the sheath 16 more than the length L of the clip series A constituted by the three clips 12A to 12C, the three connection rings 14A to 14C, the dummy clip 18, and the connection member 19 (refer to FIG. 8).

Next, by moving the slider 62 along the slider guide groove 66A to butt the slider against the end 67A, and moving the slider pin 70 to the position P2 illustrated in FIG. 11, as illustrated in FIG. 12B, the connection member 21 of the manipulating wire 20 protrudes by a predetermined amount from the forward end 16a of the sheath 16. In addition, in this embodiment, the retraction amount α₀ is included in the length A₀ of the slider guide groove 66A. Thus, even if the sheath 16 is curved, the connection member 21 can be reliably caused to protrude by a predetermined amount from the forward end 16a of the sheath 16.

In this case, for example, the clip series A is prepared in which the tail portion 24a of the turned portion 24 of the clip 12A is pinched by the claw portions 22 of the clip 12B and the engagement portion is covered with the connection ring 14A, the tail portion 24a of the turned portion 24 of the clip 12B is pinched by the claw portions 22 of the clip 12C, and the engagement portion is covered with the connection ring 14B, and the tail portion 24a of the turned portion 24 of the clip 12C is pinched by the claw portions of the dummy clip 18 and the engagement portion is covered with the connection ring 14C. Then, a clip case (not illustrated) in which the clip series is housed with the skirt portions 38 being closed is prepared.

Then, the connecting member 21 of the manipulating wire 20 is fitted into the connecting member 19 of the clip series A. Then, the slider 62 is moved along the slider guide groove 66A, and the slider pin 70 is moved to the position P3 illustrated in FIG. 11.

As a result, as illustrated in FIG. 12C, the clip series A is loaded into the sheath 16. As such, the slider guide groove 66A is used for the loading of the clip series A into the sheath 16.

Thereafter, the sheath 16 is inserted into a forceps port (not illustrated) of an endoscope (not illustrated) inserted into a living body. Then, the forward end of the sheath 16 is caused to reach the forward end of the insert portion of the endoscope, and is then caused to protrude from the forward end of the endoscope.

Additionally, if required, the forward end 16a of the sheath 16 of the clipping treatment device 10 is moved to a target position through manipulation of the insert portion or the angle portion of the endoscope.

After required manipulation is completed, next, the slider guide 66 is rotated by, for example, 90°, and as illustrated in FIG. 11, the slider pin 70 is caused to coincide with the position of the slider guide groove 66B, and is moved to the position P4.

Next, the slider 62 is moved along the slider guide groove 66B and butts the end 67B, whereby the slider pin 70 is moved to a position P5 (standard protruding position) illustrated in FIG. 11. Through this push-out of the slider 62, that is, the push-out of the manipulating wire 20, the clip series A moves to the forward end side, and as illustrated in FIG. 12D, the foremost clip 12A and the first region 32 of the connection ring 14A protrudes from the forward end 16a of the sheath 16. As a result, the arm portions 28 of the clip 12A are opened and the claw portions 22 are automatically separated from each other by a predetermined distance. Moreover, the skirt portions 38 of the connection ring 14A are opened outward more than the inner diameter of the sheath 16. As a result, the clip 12A does not return to the inside of the sheath 16, and is retained at the forward end 16a of a sheath 16 by the second region 34.

Here, in this embodiment, the retraction amount α₁ is included in the length A₁ of the slider guide groove 66B. Thus, even if the sheath 16 is curved, can be reliably caused to protrude from the forward end 16a of the sheath 16 up to a position where the skirt portions 38 of the connection ring 14A are opened. Moreover, since the connection ring 14 is provided with the extension portions 44, the clip 12A coming off is prevented even if the protruding amount is large.

Next, for example, while an image of the endoscope is viewed, the endoscope is manipulated to press the claw portions 22 of the diverged clip 12A against the position of the living body b to be clipped, and the slider 62 is moved to the proximal end side by a clipping stroke, that is, pulled back to a position P6 (a clipping complete position).

At this time, the skirt portions 38 of the connection ring 14A protruding from the forward end 16a of the sheath 16 are opened, and the retention of the first clip 12A by the pressure of the skirt portions 38 is cancelled. Additionally, since the skirt portions 38 are opened at the forward end 16a of the sheath 16, the connection ring 14A is prevented from retreating into the sheath 16. Therefore, the foremost first clip 12A retreats with respect to the connection ring 14A. The clamping ring 40 of the forward end of the connection ring 14A is pushed in to directly below the projections 30 from the crossing portion 26 side along the proximal portions 28b of the arm portions 28 of the first clip 12A, whereby as illustrated in FIG. 12E, the claw portions 22 and 22 clip the living body b, such as a disease part, and clamping of the first clip 12A by the clamping ring 40 of the forward end of the connection ring 14A is completed.

At the same time, the engagement portion between the first clip 12A and the next second clip 12B comes off from the rear ends of the extension portions 44 and 44 of the connection ring 14A. When the engagement portion between the first clip 12A and the second clip 12B is detached from the extension portions 44 and 44 of the connection ring 14A, the arm portions 28 are diverged by the resilient force of the second clip 12B until the arm portions hit the inner surfaces of the sheath 16, and the claw portions 22 and 22 are opened until the interval therebetween becomes larger than the width of the turned portion 24 of the first clip 12A, thereby canceling the connection between the first clip 12A and the second clip 12B.

As a result, as illustrated in FIG. 12F, the first clip 12A which has clipped the living body b, and the connection ring 14A is detached from the forward end 16a of the sheath 16, and the clipping treatment of a diseased part by the first clip 12A and the clamping ring 40 of the forward end of the connection ring 14A is completed.

After the clipping treatment by the first clip 12A is completed, as illustrated in FIG. 11, the slider 62 is moved to HP. That is, the slider pin 70 is moved to a position P7 of FIG. 11. As a result, as illustrated in FIG. 12G, the clip 12B and the clip 12C are brought into a state where the clips are retracted into the interior of the sheath 16. At this time, the number of clips of the clip series A decreases by one, and the number of clips in the sheath 16 is two. For this reason, the resistance against the manipulating wire 20 becomes small, and the position U₂ of the clip 12B after clipping treatment is performed by the first shot clip 12A may retraction closer to the proximal end side than the position U₁ (refer to FIG. 12C) of the clip 12B when the clip series A is loaded.

However, in this embodiment, since the length A₂ of the slider guide groove 66C is set in consideration of such retraction. Therefore, the clip 12B can be reliably caused to protrude from the forward end 16a of the sheath 16 to a position where the skirt portions 38 of the connection ring 14B are opened as will be described below.

Next, the slider guide 66 is rotated by, for example, 90°, and the slider pin 70 is caused to coincide with the position of the slider guide groove 66C, and is moved to a position P8 illustrated in FIG. 11. As a result, is moved to the HP corresponding to the slider guide groove 66C. At this time, the position of the second clip 12B is still a position illustrated in FIG. 12G.

Then, the slider 62 is moved along the slider guide groove 66C, and the slider pin 70 butts the forward end portion 67C of the slider guide groove 66C. That is, the slider pin is moved to a position P9 (standard protruding position) illustrated in FIG. 11. At this time, when the slider pin is moved to the position P9, the connection ring 14B of the clip 12B protrudes from the forward end 16a of the sheath 16, and the skirt portions 38 are is brought into an opened state. As a result, the clip 12B is brought into a preparation state for clipping.

Thereafter, the claw portions 22 of the diverged second clip 12B is pressed against the position of the living body b to be clipped, and the slider 62 is moved to the proximal end side by a clipping stroke, that is, pulled back to a position 210 (a clipping complete position), thereby completing clipping. Then, the slider 62 is moved to the proximal end side, and the slider pin 70 is moved to a position P11.

At this time, since only the clip 120 is present in the sheath 16, the resistance against the manipulating wire 20 becomes smaller, and the position of the clip 12C after clipping treatment is performed by the first shot clip 12A and the second shot clip 12B may retraction closer to the proximal end side than the position of the clip 12C when the clip series A is loaded.

However, even in this case, similarly to the clip 12B, in this embodiment, the length A₃ of the slider guide groove 66D is set in consideration of such retraction. Therefore, the clip 12C can be reliably caused to protrude from the forward end 16a of the sheath 16 to a position where the skirt portions 38 of the connection ring 14C are opened as will be described below.

Next, the slider guide 66 is rotated by 90°, and the slider pin 70 is caused to coincide with the position of the slider guide groove 66D, and is moved to a position P12.

Then, the slider 62 is moved to the forward end side along the slider guide groove 66D, and the slider pin 70 is moved to a position where the slider pin comes into contact with the forward end portion 67D of the slider guide groove 66D. That is, the slider pin is moved to a position P13 (standard protruding position) illustrated in FIG. 11. At this time, when the slider pin is moved to the position P13, the connection ring 14C of the clip 12C protrudes from the forward end 16a of the sheath 16, and the skirt portions 38 are is brought into an opened state. As a result, the clip 12C is brought into a preparation state for clipping.

Thereafter, the claw portions 22 of the diverged clip 12C is pressed against the position of the living body b to be clipped, and the slider 62 is moved to the proximal end side by a clipping stroke, that is, pulled back to a position P14 (a clipping complete position), thereby completing clipping. Then, the slider 62 is moved to the proximal end side, and the slider pin 70 is moved to a position P15. As a result, the clipping using the clipping treatment device 10 is completed.

If the clipping by three clips 12A to 12C is completed, the slider 62 is rotated by 90° toward the slider guide groove 66A side, and returns to HP corresponding to the position P1 of FIG. 11, that is, the slider guide groove 66A. Thereafter, the sheath 16 of the clipping treatment device 10 is pulled out of the endoscope.

After the sheath 16 has been pulled out, the slider 62 is moved along the slider guide groove 66A and pushed out to the position P2, the connection member 21, the dummy clip 18 and the connecting member 19 are caused to protrude from the forward end 16a of the sheath 16, and the dummy clip 18 and the connecting member 19 are caused to protrude from the connection member 21 of the forward end of the manipulating wire 20.

The clipping can be performed using the clipping treatment device 10 of this embodiment as described above.

Additionally, in this embodiment, when the manipulating wire 20 has elongated for a prolonged period of use, adjustment is made as follows. The lock dial 64 is first loosened and the adjustment dial 60 is then turned, whereby the adjustment dial 60 is moved to the forward end side with respect to the wire fixing member 58. Since the slider 62 is engaged with the projection of the adjustment dial 60, the adjustment dial moves in the axial direction together with the slider 62. Here, since the movement of the adjustment dial 60 and the slider 62 in the rotating direction is free, the circumferential movement of the slider 62 is regulated by the slider pin 70, whereas only the adjustment dial 60 can be rotated. If the manipulating wire 20 has been moved enough that the loosening of the adjustment dial 60 is eliminated, the lock dial 64 is fastened again to fix the positions of the adjustment dial 60 and the slider 62.

Since the home position of the slider 62 is regulated as the lock dial 64 comes into contact with the cylindrical portion 52a of the main body rail 52, the position of the slider 62 with respect to the main body rail 52 at the home position does not change. On the other hand, by moving the adjustment dial 60 and the slider 62 to the forward end side with respect to the wire fixing member 58, the wire fixing member 58 moves rearward relative to the slider 62, and the wire connecting portion 58a moves similarly. Accordingly, the proximal end of the manipulating wire 20 at the home position moves rearward, and the loosening equivalent to the elongation of the manipulating wire 20 can be canceled.

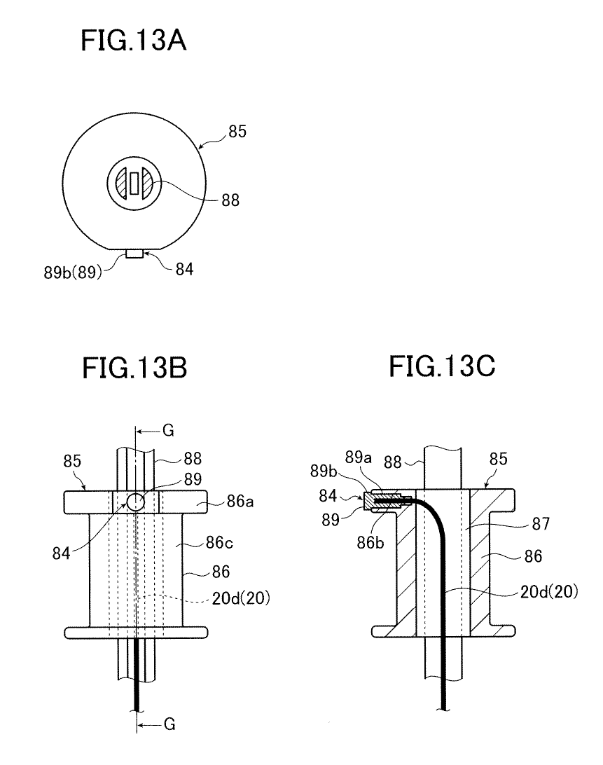

Next, an adjusting mechanism which finely adjusts the position of the manipulating wire 20 with respect to the sheath 16 will be described with reference to FIGS. 13A to 13C.

FIG. 13A is a schematic view illustrating an example of the adjusting mechanism of the manipulating wire of the handle portion of the manipulation handle of the repetitive clipping treatment device of the embodiment of the invention, FIG. 13B is a plan view illustrating the example of the adjusting mechanism of the manipulating wire of the handle portion of the manipulation handle of the repetitive clipping treatment device of the embodiment of the invention, and FIG. 13C is a cross-sectional view taken along the line G-G of FIG. 13B.

In addition, since the configuration other than the adjusting mechanism 84 of the manipulating wire 20 and the slider 85 are the same as the manipulating portion 50 illustrated in FIG. 5, the detailed description thereof is omitted.

The slider 85 has a substantially cylindrical slider main body 86, and an inner member 87 fixed to the insider thereof, and is movable along the main body rail 88 as a whole. The manipulating wire 20 is inserted through the inside of the reinforcing pipe 20d in the vicinity of the slider 85. The reinforcing pipe 20d extends along the axis of the main body rail 88 and is fixed to the inner member 87, and the proximal end portion thereof is curved, is changed in direction by 90°, and extends in the radial direction of the larger-diameter portion 86a of the slider main body 86. The proximal end portion of the manipulating wire 20 inserted through the reinforcing pipe 20d is exposed from the proximal end of the reinforcing pipe 20d, and the exposed portion is fixed to the adjustment dial 89. The adjustment dial 89 is disposed toward the center of the larger-diameter portion 86a, and the outer peripheral surface thereof is formed with a male thread 89a. A cylindrical recess is formed in the larger-diameter portion 86a from the outer peripheral surface toward the center, and the male thread 89a of the adjustment dial 89 is screwed to a female thread 86b formed in the inner peripheral surface of this recess. The adjusting mechanism 84 is constituted by the adjustment dial 89, the female thread 86b of the slider main body 86, the reinforcing pipe 20d, and the like.

According to the adjusting mechanism 84, when the operator pinches the adjustment dial 89 between his/her thumb and index finger, for example, rotates the adjustment dial in the clockwise direction, the adjustment dial 89 proceeds toward the center of the larger-diameter portion 86a, and the manipulating wire 20 integral with the adjustment dial 89 is pushed out and the forward end side thereof moves to the proximal end side in the axial direction. On the other hand, when the operator rotates the adjustment dial 89 in the opposite counterclockwise direction, the adjustment dial 89 proceeds in a direction away from the center of the larger-diameter portion 86a, and the manipulating wire 20 integral with the adjustment dial 89 is pulled and the forward end side thereof moves to the proximal end side in the axial direction.

Since the adjustment dial 89 uses a screw mechanism, the axial movement of the adjustment dial to the displacement thereof in the rotating direction is small. That is, the axial displacement on the forward end side of the manipulating wire 20 whose proximal end portion is connected to the adjustment dial 89 can be finely adjusted by rotating the adjustment dial 89 in the clockwise direction or in the counterclockwise direction.

According to the adjusting mechanism 84, since the fingertip of the index finger when the slider 85 is manipulated to advance or retreat can be placed near a head 89b of the adjustment dial 89, the operability of the adjustment dial 89 can be enhanced. That is, when the slider 85 is manipulated, particularly, when the slider 85 is retreated to pull the manipulating wire 20, the operator nips a smaller-diameter portion 86c of the slider main body 86 between his/her index finger and middle finger, and the operator applies the insides of the fingertips of the two fingers to the forward end surface of the proximal-end-side larger-diameter portion 86a, and pulls the larger-diameter portion 86a to the front side (thumb side). Accordingly, when the operator manipulates the slider 85, the operator can positively apply his/her index finger so as to contact the vicinity of the head 89b of the adjustment dial 89 and further the head 89b directly. According to this, when the operator turns the adjustment dial 89 to finely adjust the manipulating wire 20, subsequent to the advance/retreat manipulation of slider 85, the operator simply removes his/her thumb from the finger hook ring 56 (refer to FIG. 5) to hook his/her thumb on the head 89b of the adjustment dial 89, without moving his/her index finger and middle finger, so that the operator can nip the head 89b with his/her index finger and thumb, and rotate the adjustment dial 89 to finely adjust the manipulating wire 20. That is, the axial push-pull (axial large movement) of the manipulating wire 20 by the advance/retreat manipulation of a slider 85 and the fine adjustment (axial small movement) of the manipulating wire 20 by the rotation manipulation of the adjustment dial 89 can be smoothly performed as a series of manipulations without wasted movement of the fingertip.

In addition, if the diameter of the head 89b of the adjustment dial 89 is made larger than the axial thickness of the larger-diameter portion 86a of the slider main body 86, and portions of the head 89b are caused to protrude from the proximal end surface and forward end surface of the larger-diameter portion 86a, these protruding portions reliably hit the insides of the fingertips of the index finger and the thumb. Thus, operability can be further improved. Moreover, it is also possible to adopt, for example, a tapered shape or a curved shape taken after the shape of the belly of the fingertip of the index finger instead of the columnar shape as the shape of the outer peripheral surface of the head 89b. In this case, when the operator grasps (nips) the slider main body 86, the operator can touch the head 89b of the adjustment dial 89 with high/her index finger, thereby positioning the finger during manipulation at the home position.

In addition, even in the adjusting mechanism 84, home positioning may be performed by forming home position marks in the proximal-end-side larger-diameter portion 86a of the slider main body 86 and the head 89b of the adjustment dial 89, respectively, and aligning the larger-diameter portion with the head.

Even in the adjusting mechanism 84, it is preferable to provide a loosening stop mechanism equivalent to the lock dial 64 illustrated in FIG. 5. Here, for example, when a configuration in which a frictional force with the manipulating wire 20 increases is provided at the curved portion of the reinforcing pipe 20d, it is also possible to adopt this as the loosening stop mechanism.

Although the repetitive clipping treatment device of the invention have been described in detail above, the invention is not limited to the above embodiment, and it is needless to say that various improvements and modifications are possible without departing from the spirit of the invention.

### REFERENCE SIGNS LIST

10 clipping treatment device (repetitive clipping treatment device)
12, 12A to 12C clip
14, 14A to 14C connection ring
16 sheath
18 dummy clip
21 connection member
20 manipulating wire
22 claw portion
24 turned portion
38 skirt portion
40 clamping ring
44 extension portion
48 manipulation handle
50 manipulating portion
52 main body rail
56 finger hook ring
58 wire fixing member
60 adjustment dial
62 slider
64 lock dial
66 slider guide (slider displacement regulating member)
66a to 66d slider guide groove
68 position regulating member
70 slider pin
72 reinforcing pipe
76 grasping portion

1. A repetitive clipping treatment device comprising:
a clip series having plural clips connected by a forward end of a subsequent clip being engaged with a rear end of a previous clip, and a connection member connected to the rearmost clip;
a cylindrical sheath loaded with the clip series;
a manipulating wire movably arranged in the sheath, and having a forward end detachably connected to the connection member so as to move the clip series in a longitudinal direction of the sheath in the sheath;
a handle main body connected to the sheath and having the manipulating wire extending from the sheath arranged therein;
a slider mounted on an outer periphery of the handle main body so as to be movable in an axial direction of the handle main body, engaged with the manipulating wire, and moving the manipulating wire in the axial direction of the handle main body, and
a slider displacement regulating member mounted on the outer periphery of the handle main body so as to be rotatable in a circumferential direction of the handle main body and regulating displacement of the slider in an axial direction of the handle main body to plural different displacements required for clipping treatment of each clip of the clip series,
wherein plural different displacements regulated by the slider displacement regulating member and corresponding to respective clips is calculated by obtaining an amount of retraction at a position of a clip of the clip series in advance, and determining a displacement based on a length required to cause each clip to protrude, and respective retraction amounts in each clip.

2. The repetitive clipping treatment device according to claim 1,
wherein an engagement portion between the previous clip and the subsequent clip in the clip series is covered with a connection ring fitted so as to be able to advance or retreat with respect to the clips, and
wherein the connection ring is pressed against an inner surface of the sheath and closed inward in the sheath, and presses and retains at least one of the clips connected in the connection ring, and at least two or more skirt portions opened more widely than the inner diameter of the sheath after passing through a forward end of the sheath to prevent retreating to an inside of the sheath are provided in a circumferential direction of the connection ring at the same position in a movement direction of the clips.

3. The repetitive clipping treatment device according to claim 2,
wherein an extension portion is provided as a portion of the connection ring on a proximal end side and extends, and the extension portion prevents a clip and the connection ring from coming off of the forward end of the sheath during clipping treatment.

4. The repetitive clipping treatment device according to claim 1,
wherein the slider displacement regulating member has plural position regulating grooves which is formed according to the plural different displacements, respectively, at predetermined intervals in the circumferential direction of the handle main body, and have different positions at forward end portions thereof on the sheath side.

5. The repetitive clipping treatment device according to claim 4,
wherein the slider displacement regulating member has position regulating grooves equal to or more than the number of the clips provided in the clip series.

6. The repetitive clipping treatment device according to claim 4,
wherein the slider displacement regulating member is arranged so that a position regulating groove whose sheath-side forward end portion is nearest to the sheath, and a position regulating groove whose forward end portion is farthest from the sheath are adjacent to each other, and
wherein a position regulating groove formed on the side where the position regulating groove whose forward end portion is nearest to the sheath, and the position regulating groove whose forward end portion is farthest from the sheath are not adjacent to each other is formed so that the forward end portion thereof is located at a position near the sheath gradually, as it goes from the position regulating groove whose forward end portion is nearest to the sheath, and the position regulating groove whose forward end portion is farthest from the sheath, on the side where the position regulating groove whose forward end portion is nearest to the sheath, and the position regulating groove whose forward end portion is farthest from the sheath are not adjacent to each other.

7. The repetitive clipping treatment device according to claim 6,
wherein the position regulating groove whose forward end portion is nearest to the sheath in the slider displacement regulating member is used for loading of the clip series into the sheath.

8. The repetitive clipping treatment device according to claim 4,
wherein the slider has a guide pin which extends toward the center of the handle main body and has a diameter capable of being inserted through the position regulating grooves, and
wherein, when the position of one position regulating groove of the plural position regulating grooves overlaps the position of the guide pin, the slider (the slider) moves in the axial direction of the handle main body along the overlapped position regulating groove.

9. The repetitive clipping treatment device according to claim 4,
wherein the clip series includes three clips, and the slider displacement regulating member has the position regulating grooves at intervals of 90° in the circumferential direction of the handle main body.

10. The repetitive clipping treatment device according to claim 1,
wherein the clip has at least a claw portion and an arm portion, and the arm portion has a projection.

11. The repetitive clipping treatment device according to claim 10,
wherein the projection is wider than the arm portion.
